(19) United States
(12) Patent Application Publication
(10) Pub. No.: US 2024/0087679 A1
(43) Pub. Date: Mar. 14, 2024

(54) SYSTEMS AND METHODS OF VALIDATING NEW AFFINITY REAGENTS

(71) Applicant: NAUTILUS SUBSIDIARY, INC., Seattle, WA (US)

(72) Inventors: Jarrett D. EGERTSON, Rancho Palos Verdes, CA (US); Parag MALLICK, San Mateo, CA (US)

(21) Appl. No.: 18/463,100

(22) Filed: Sep. 7, 2023

Related U.S. Application Data
(60) Provisional application No. 63/375,514, filed on Sep. 13, 2022.

(51) Int. Cl. G16B 20/30; C12Q 1/6834

(52) U.S. Cl. CPC G16B 20/30; C12Q 1/6834

(57) ABSTRACT

Disclosed are systems and methods for identifying the binding characteristics of a partially characterized or completely unknown affinity reagent, such as an antibody or aptamer, by binding that affinity reagent against an array of known proteins. The proteins on the array which bind to the partially characterized or completely unknown affinity reagent are then determined and an analysis performed to determine the binding characteristics of the affinity reagent.

### CROSS-REFERENCE TO RELATED APPLICATIONS

This application claims priority to U.S. Provisional Application No. 63/375,514, filed on Sep. 13, 2022, which application is incorporated herein by reference.

### FIELD

The present disclosure relates to a method of validating new affinity reagents. More specifically, the present disclosure relates to systems and methods for validating the binding characteristics of a spectrum of partially characterized or completely unknown affinity reagents by binding them to characterized proteins on a proteomic array and measuring such binding.

### BACKGROUND

Current techniques for protein identification typically rely upon either the binding and subsequent readout of highly specific and sensitive affinity reagents (such as antibodies) or upon peptide-read data (typically on the order of 12-30 amino acids long) from a mass spectrometer. Such techniques may be applied to unknown proteins in a sample to determine the presence, absence, or quantity of candidate proteins based on analysis of binding measurements of the highly specific and sensitive affinity reagents to the protein of interest.

### SUMMARY

The present disclosure provides method and system for analyzing, measuring and validating new affinity reagents. This may occur while decoding a proteomic array. An aspect of the present disclosure provides a method of identifying binding characteristics of a spectrum of test affinity reagents, such as affinity reagents that are partially characterized or completely unknown. The method may also include observing which proteins are not bound by the affinity reagent. The method comprises providing a substrate with a plurality of attached proteins corresponding to a portion of a proteome.

In some embodiments, one or more signals are produced by the spectrum of test affinity reagents (e.g. partially characterized or completely unknown affinity reagents) when determining the one or more spatial addresses on the substrate that a spectrum of test affinity reagents are bound to. In some cases, the unknown affinity reagent is configured to bind to one particular epitope of a portion of the plurality of the proteins on an array. In some instances, the unknown affinity reagent is specific to an individual and distinguishable protein or protein family.

In some cases, the test affinity reagents (e.g. partially characterized or completely unknown affinity reagents) recognize a particular epitope which is present in one or more of the plurality of proteins. In some cases, the particular epitope is a conformational epitope. In some instances, the particular epitope is a linear epitope. In some cases, the affinity reagent binds to a particular sequence of contiguous amino acids that is unique to the particular epitope. In some cases, the affinity reagent binds to a set of non-contiguous amino acids which is unique to a specific epitope on a protein. In some cases, the affinity reagent is an antibody which binds to a particular trimer amino acid sequence.

In some embodiments, the method further comprises additional steps taken by the system to determine the probability that certain proteins on the array will be bound by the affinity reagents to a predetermined threshold degree of accuracy. This information can be used to train a machine learning model which can predict the degree of binding (e.g.: probability of binding and of non-binding) of the affinity reagents to a protein or polypeptide given the protein's amino acid sequence. Once the machine learning model is sufficiently predictive based on its training then these test affinity reagents (e.g. partially characterized or completely unknown affinity reagents) can be used as known affinity reagents in later experiments.

In some instances, the substrate is a flow cell. In some instances, the proteins are attached to the substrate using a photo-activatable linker. In some instances, the proteins are attached to the substrate using a photo-cleavable linker.

In some instances, the test affinity reagents (e.g. partially characterized or completely unknown affinity reagents) are modified from their endogenous form to be conjugated to an identifiable tag. In some instances, the identifiable tag is a fluorescent tag. In some instances, an identifiable tag is a nucleic acid barcode. In some instances, a binding characteristic of the test affinity reagent is identified using deconvolution software. In some instances, identifying a binding characteristic of the test affinity reagent is determined by decoding binding measurements associated with unique spatial addresses bound by the test affinity reagents.

In some instances, the proteins are isolated from a biological source. In some cases, the proteins are attached to random addresses on the substrate prior to identifying one or more binding sites for each patterned protein.

Another embodiment is a method of characterizing a test affinity reagent (e.g. partially characterized affinity reagent) using a machine learning model. The method includes: providing a substrate having a plurality of attached proteins corresponding to at least a portion of a proteome, wherein each attached protein has a unique spatial address on the substrate, wherein the identity of the protein at each said spatial address is unknown; determining the identity of the proteins at each spatial address by 1) applying a set of known affinity reagents to the substrate and measuring whether the known set of affinity reagents binds, or does not bind to the attached proteins and 2) identifying the proteins according to the machine learning model; applying a test affinity reagent (e.g. partially characterized affinity reagent) to the substrate; determining the one or more spatial addresses where the test affinity reagent binds, and does not bind, to the substrate; and inputting the binding characteristics of the test affinity reagent to the trained machine learning model to characterize the test affinity reagent.

Still another embodiment is a system for characterizing a test affinity reagent (e.g. partially characterized affinity reagent) using a machine learning model. This embodiment includes: a substrate having a plurality of proteins corresponding to a portion of a proteome bound thereto, wherein one or more binding sites for each bound protein has been identified and each bound protein has a unique, optically resolvable, spatial address on the substrate; and a processor configured to execute instructions that when run on the processor perform the method of: determining the identity of the proteins at each spatial address by 1) applying a set of known affinity reagents to the substrate and measuring whether the known set of affinity reagents binds, or does not bind to the attached proteins and 2) identifying the proteins according to the machine learning model; applying a partially characterized affinity reagent to the substrate; determining the one or more spatial addresses where the test affinity reagent binds, and does not bind, to the substrate; and inputting the binding characteristics of the test affinity reagent to the trained machine learning model to characterize the test affinity reagent.

One additional embodiment is a method of identifying binding characteristics of a test affinity reagents (e.g. partially characterized or completely unknown affinity reagent) using a machine learning model. This method includes: providing a substrate having a plurality of attached proteins, wherein the identity of the proteins at each position has been determined by identifying the binding of a known set of affinity reagents to the sub state and the binding characteristics of each affinity reagent were calculated according to the machine learning model; applying a test affinity reagent to the substrate; and inputting the binding characteristics of the test affinity reagent to the machine learning model to identify the binding characteristics of the test affinity reagent.

Also provided is a method of profiling an affinity reagent. The method can include steps of (a) providing a substrate having a plurality of attached proteins corresponding to at least a portion of a proteome, wherein each attached protein has a unique spatial address on the substrate, wherein the identity of the protein at each said spatial address is unknown; (b) determining the identity of the protein at each spatial address; (c) testing affinity reagents from a sample by (i) applying an affinity reagent from the sample to the substrate under a first condition, (ii) determining the one or more spatial addresses where the affinity reagent binds under the first condition, and optionally determining one or more spatial addresses where the affinity reagent does not bind, and (iii) repeating (i) and (ii) under a second condition instead of the first condition, the second condition differing from the first condition, wherein the affinity reagent tested under the first condition has identical composition to the affinity reagent tested under the second condition; and (d) determining a binding characteristic of the affinity reagent based on the testing of the affinity reagents from the sample. Optionally, (iii) can include repeating (i) and (ii) a plurality of times, each time under a different condition, wherein the affinity reagent tested under the different conditions has identical composition to the affinity reagent tested under the first condition.

The present disclosure further provides a method of characterizing a protein ligand. The method can include steps of (a) providing a substrate having a plurality of attached proteins corresponding to at least a portion of a proteome, wherein each attached protein has a unique spatial address on the substrate, wherein the identity of the protein at each said spatial address is unknown; (b) determining the identity of the protein at each spatial address; (c) applying a ligand to the substrate; (d) determining one or more spatial addresses where the ligand binds, and optionally, determining one or more spatial addresses where the ligand does not bind; and (e) identifying at least one protein on the array to which the ligand binds.

Further provided is a method of characterizing a protein reactant. The method can include steps of (a) providing a substrate having a plurality of attached proteins corresponding to at least a portion of a proteome, wherein each attached protein has a unique spatial address on the substrate, wherein the identity of the protein at each said spatial address is unknown; (b) determining the identity of the protein at each spatial address; (c) applying a reactant to the substrate; (d) determining one or more spatial addresses having a protein that is modified by the reactant, and optionally determining one or more spatial addresses having a protein that is not modified by the reactant; and (e) identifying at least one protein on the array that is modified by the reactant.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 illustrates a first schematic of protein quantification by anti-protein antibody decoding, in accordance with some embodiments. Panel a) shows sample preparation and protein deposition on an array. Panel b) shows unknown affinity reagent hybridization and imaging. Panel c) shows data processing to identify the bound unknown affinity reagent.

FIG. 2 illustrates a flow diagram of one method of performing a method of characterizing partially characterized or completely unknown affinity reagents according to embodiments of the present disclosure.

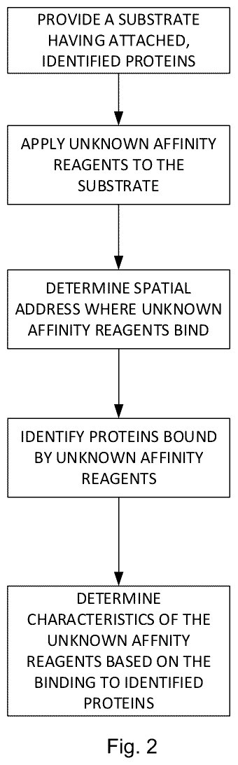

FIG. 3 illustrates a computer control system that is programmed or otherwise configured to implement methods provided herein.

### DETAILED DESCRIPTION

### Overview

Embodiments relate to systems and methods for determining the makeup of a proteome from a biological source such as a biological fluid, cell or tissue. In some aspects the proteins expressed within a biological source are isolated from the source and then bound to an array, with a single protein being bound in each spatial address of the array. A labeled set of known affinity reagents, such as labeled antibodies, can be used in a first cycle to identify the proteins bound at each address. For example, known affinity reagents which are known to bind to particular epitopes (e.g. an epitope can be a short amino acid sequence) can be repeatedly bound to the array to determine the protein sequence, or partial protein sequence of each protein on the array. Then, the binding (and, optionally non-binding) patterns of affinity reagents can be used in conjunction with a decoding system to identify known. Exemplary systems are described in U.S. Pat. Nos. 10,473,654 or 11,282,585, or Egertson et al., Biokciv (2021), DOI: 10.1101/2021.10.11.463967, each of which is hereby incorporated by reference in its entirety.

Embodiments relate to methods and systems for characterizing affinity reagents where the goal of characterization is to be able to predict the probability of the affinity reagent binding to a protein given the primary amino acid sequence of that protein. These methods and systems may use machine learning/artificial intelligence to develop models which may predict the probability of whether a particular affinity reagent will bind to a protein sequence. In a first embodiment, the method may start with a protein array which has a plurality of individual known proteins bound at known addresses on the array. Test affinity reagents (e.g. unknown or partially characterized affinity reagents) are then bound to the known proteins on the array and each affinity reagent is analyzed to determine if it bound, or did not bind, to each protein on the array. That binding (and, optionally non-binding) data is input into a machine learning system to develop a model which can be used to determine the probability that the unknown affinity reagent will bind to proteins having the amino acid sequences of the proteins on the array. In a second embodiment, a plurality of unknown proteins is bound to an array, and then a series of characterized affinity reagents are serially bound to the array. This data is then input into a machine learning system that determines the identity of the unknown proteins at the addresses on the array based on a model for calculating the probability that the characterized affinity reagents will bind to candidate proteins expected to be on the array.

In the second embodiment, the proteins on the array are unknown initially, but since the affinity reagents being used are known to bind particular proteins, or amino acid sequences, the machine learning system can use a model based on binding of the known affinity reagents to the unknown proteins on the array, to determine the protein that is present at each address on the array. The method can then proceed as set forth above for the first embodiment to bind test affinity reagents (e.g. unknown or partially characterized affinity reagents) to the array and input binding results into the machine learning system to determine the probability that the unknown affinity reagent will bind proteins having the amino acid sequences of the proteins on the array. It will be understood that the known affinity reagents need not be contacted with the array prior to contacting the array with the test affinity reagents. Rather, some or all of the test affinity reagents can be contacted with the array prior to completing the process of contacting the known affinity reagents with the array, and so long as the results for each affinity reagent are distinguished from the results of every other affinity reagent, the results for the two types of affinity reagents can be processed by the machine learning system as set forth herein.

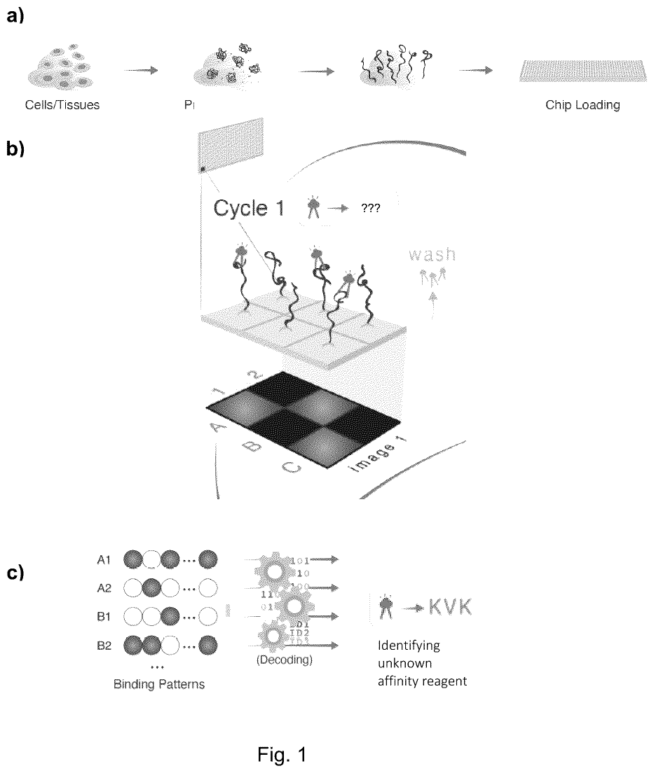

Some embodiments provided herein relate to a system and process for characterizing new affinity reagents where their binding characteristics and targets are unknown or partially characterized, which may be useful for improving the suite of affinity reagents available for future procedures. For example, as shown in FIG. 1, panel a) in some aspects the proteins expressed within a cell or tissue are isolated from the cells or tissue and then bound to an array, with a single protein being bound in each spatial address of the array. As shown in FIG. 1, panel b) a first cycle (Cycle 1) is run where labeled, test affinity reagents (e.g. partially characterized or completely unknown affinity reagents) are bound to the proteins on the array, the identity of the protein at each particular address on the array is assigned, with a level of confidence. It should be realized that copies of a particular protein may be located at multiple addresses on the array. The binding addresses of each test affinity reagent on the array may be determined from the fluorescent pattern captured of the bound affinity reagent. Alternatively, during one run in which a proteomic array is contacted with a plurality of affinity reagents that are known to bind to particular epitopes, the run can also include one or more affinity reagents where their target epitopes are unknown. The affinity reagents with unknown target epitopes sites will bind to certain proteins on the array, and their binding characteristics will be captured by the system. As shown in FIG. 1, panel c), once the system decodes the identity of each protein on the array from the binding and, optionally non-binding events of the known affinity reagents, a determination can be made of which proteins were bound by each unknown affinity reagent. This information can be used to train a machine learning model which can predict the degree of binding (e.g.: probability of binding and, optionally of non-binding) of the affinity reagents to a protein or polypeptide given its amino acid sequence. Once the machine learning model is sufficiently predictive based on its training then these test affinity reagents can then be used as known affinity reagents.

In some embodiments, the affinity reagents can be given a score to rank how well characterized they are in comparison to fully characterized affinity reagents. For example, each affinity reagent may be given a score from 0-1, where 0 indicates an affinity reagent with no characterization data, and a score of 1 indicates a fully characterized and known affinity reagent.

In some examples, embodiments can include an approach comprising three aspects: 1) an addressable substrate to which proteins and/or protein fragments are conjugated; 2) a test affinity reagents (e.g. partially characterized or completely unknown affinity reagent), e.g. where each affinity reagent can bind to a peptide with varying specificity; and 3) software and hardware that is able to use a combination of prior knowledge about the binding characteristics of known affinity reagents, the specific pattern of binding and, optionally non-binding of the unknown affinity reagent at each address in the substrate, and/or a database of the sequences of the proteins in the mixture (e.g. the human proteome) to infer the identity of the test affinity reagent at a precise spatial address in the substrate. In some examples, the precise spatial address may be a unique spatial address.

Another embodiment includes a process of identifying the binding characteristics of a test affinity reagent (e.g. partially characterized affinity reagent). In one example of this process, an affinity reagent designed to predominantly bind to a particular trimer amino acid sequence is applied to a first array comprising attached peptides to partially characterize the binding and, optionally non-binding aspects of the affinity reagent. In one embodiment, the first array may be manufactured using the PEPperPrint™ platform where known proteins or peptides are bound to addresses at known locations in an array. The process then moves to a state wherein the affinity reagent is evaluated based on whether the affinity reagent bound, or didn't bind, to one or more of the known proteins. More specifically, the binding measurements of the affinity reagent and the known proteins or peptides on the array are input into a machine learning system to train a binding model to determine the probability that the affinity reagent will bind to a certain protein. The characterized affinity reagent is then applied to a second array substrate comprising proteins from a proteome. The spatial addresses where the affinity reagent binds (and optionally where it does not bind) to the second array substrate are then determined. The process obtains the identity of the proteins on the second array by applying a plurality of known affinity reagents and by applying a machine learning model that has been trained to identify proteins based on the probabilities that the known affinity reagents bind to particular protein sequences. The process obtains the initial binding characteristics of the affinity reagents from the binding and, optionally non-binding events to the first array. Those characteristics and the binding results observed for the second array can then be input to the trained machine learning model to further characterize the affinity reagent.

Still another embodiment relates to a process of identifying the binding characteristics of affinity reagents by binding them to a first protein array to gain characterization data for the affinity reagent, and then applying the affinity reagent to a second proteomic array to perform one or more cycles of decoding to generate some partially characterized protein binding data for which the identity is known for only a subset of the proteins. The partially characterized protein data may undergo one or more alternating cycles of protein decoding followed by machine learning training to generate more data to provide a better characterized set of affinity reagents. In some embodiments, a machine learning/artificial intelligence learning process may be used to help characterize the affinity reagents. The partially characterized protein data may enhance the machine learning process to result in a better decoding process and more accurate determination of binding characteristics of the affinity reagent.

A characteristic determined for a test affinity reagent (e.g. partially characterized or completely unknown affinity reagent) can include a measure of affinity, for example, a probability of a positive binding outcome with a particular protein or a probability of a negative binding outcome with a particular protein. A characteristic determined for a test affinity reagent (e.g. partially characterized or completely unknown affinity reagent) can include a measure of promiscuity or non-specificity, for example, probabilities of positive binding outcomes with a plurality of different proteins, or a probability of non-specific binding to a substrate such as a substrate for an array of proteins. A binding model can be determined for the test affinity reagent to include one or more of the probabilities. Accordingly, a binding model can include a function for determining probability of a positive binding outcome occurring between an affinity reagent and one or more proteins, or other objects. Alternatively, or additionally, a binding model can include a function for determining probability of a negative binding outcome occurring between an affinity reagent and one or more proteins, or other objects.

### Protein Samples

As used herein, the samples may be any sample containing protein, for example, a biological sample or synthetic sample. The samples may be taken from tissue or cells or from the environment of tissue or cells. In some cases, the sample could be a tissue biopsy, blood, blood plasma, extracellular fluid, cultured cells, culture media, discarded tissue, plant matter, synthetic proteins, archael, bacterial and/or viral samples, fungal tissue, archaea, or protozoans. In some other cases, the protein is isolated from its primary source (cells, tissue, bodily fluids such as blood, environmental samples etc.) during sample preparation. The protein may or may not be purified from its primary source. In some cases, the primary source is homogenized prior to further processing. In some cases, cells are lysed using a buffer such as RIPA buffer. Proteins may optionally be denatured at any stage of extraction and separation for example using denaturing buffers. The sample may be processed, for example, being filtered or centrifuged, to remove lipids and particulate matter. The sample may also be purified to remove nucleic acids, or may be treated with RNases and DNases. The sample may contain intact proteins, denatured proteins, protein fragments or partially degraded proteins.

The sample may be taken from a subject with a disease or disorder. The disease or disorder may be an infectious disease, an immune disorder or disease, a cancer, a genetic disease, a degenerative disease, a lifestyle disease, an injury, a rare disease or an age related disease. The infectious disease may be caused by bacteria, viruses, fungi and/or parasites. Non-limiting examples of cancers include Bladder cancer, Lung cancer, Brain cancer, Melanoma, Breast cancer, Non-Hodgkin lymphoma, Cervical cancer, Ovarian cancer, Colorectal cancer, Pancreatic cancer, Esophageal cancer, Prostate cancer, Kidney cancer, Skin cancer, Leukemia, Thyroid cancer, Liver cancer, and Uterine cancer. Some examples of genetic diseases or disorders include, but are not limited to, cystic fibrosis, Charcot-Marie-Tooth disease, Huntington's disease, Peutz-Jeghers syndrome, Down syndrome, Rheumatoid arthritis, and Tay-Sachs disease. Non-limiting examples of lifestyle diseases include obesity, diabetes, arteriosclerosis, heart disease, stroke, hypertension, liver cirrhosis, nephritis, cancer, chronic obstructive pulmonary disease (COPD), hearing problems, and chronic backache. Some examples of injuries include, but are not limited to, abrasion, brain injuries, bruising, burns, concussions, congestive heart failure, construction injuries, dislocation, flail chest, fracture, hemothorax, herniated disc, hip pointer, hypothermia, lacerations, pinched nerve, pneumothorax, rib fracture, sciatica, spinal cord injury, tendons ligaments fascia injury, traumatic brain injury, and whiplash. The sample may be taken before and/or after treatment of a subject with a disease or disorder. Samples may be taken before and/or after a treatment. Samples may be taken during a treatment or a treatment regime. Multiple samples may be taken from a subject to monitor the effects of the treatment over time. The sample may be taken from a subject known or suspected of having an infectious disease for which diagnostic antibodies are not available.

The sample may be taken from a subject suspected of having a disease or a disorder. The sample may be taken from a subject experiencing unexplained symptoms, such as fatigue, nausea, weight loss, aches and pains, weakness, or memory loss. The sample may be taken from a subject having explained symptoms. The sample may be taken from a subject at risk of developing a disease or disorder due to factors such as familial history, age, environmental exposure, lifestyle risk factors, or presence of other known risk factors.

The sample may be taken from an embryo, fetus, or pregnant woman. In some examples, the sample may comprise of proteins isolated from the mother's blood plasma. In some cases, proteins isolated from circulating fetal cells in the mother's blood.

Protein may be treated to remove modifications that may interfere with binding. For cases the protein may be glycosidase treated to remove post translational glycosylation. The protein may be treated with a reducing agent to reduce disulfide binds within the protein. The protein may be treated with a phosphatase to remove phosphate groups. Other non-limiting examples of post translational modifications that may be removed include acetyl group, amide groups, methyl groups, lipids, ubiquitin, myristoylation, palmitoylation, isoprenylation or prenylation (e.g. farnesol and geranylgeraniol), farnesylation, geranylgeranylation, glypiation, lipoylation, flavin moiety attachment, phosphopantetheinylation, and retinylidene Schiff base formation. Samples may also be treated to retain posttranslational protein modifications. In some examples, phosphatase inhibitors may be added to the sample. In some examples, oxidizing agents may be added to protect disulfide bonds.

Proteins may be denatured in full or in part. In some embodiments, proteins can be fully denatured. Proteins may be denatured by application of an external stress such as a detergent, a strong acid or base, a concentrated inorganic salt, an organic solvent (e.g., alcohol or chloroform), radiation or heat. Proteins may be denatured by addition of a denaturing buffer. Proteins may also be precipitated, lyophilized and suspended in denaturing buffer. Proteins may be denatured by heating. Methods of denaturing that are unlikely to cause chemical modifications to the proteins may be preferred.

Proteins of the sample may be treated to produce shorter polypeptides, either before or after conjugation. Remaining proteins may be partially digested with an enzyme such as ProteinaseK to generate fragments or may be left intact. In further examples the proteins may be exposed to proteases such as trypsin. Additional examples of proteases may include serine proteases, cysteine proteases, threonine proteases, aspartic proteases, glutamic proteases, metalloproteases, and asparagine peptide lyases.

In some cases, it may be useful to remove extremely large and small proteins (e.g. Titin), such proteins may be removed by filtration or other appropriate methods. In some examples, extremely large proteins may include proteins that are over 400 kD, 450 kD, 500 kD, 600 kD, 650 kD, 700 kD, 750 kD, 800 kD or 850 kD. In some examples, extremely large proteins may include proteins that are over about 8,000 amino acids, about 8,500 amino acids, about 9,000 amino acids, about 9,500 amino acids, about 10,000 amino acids, about 10,500 amino acids, about 11,000 amino acids or about 15,000 amino acids. In some examples, small proteins may include proteins that are less than about 10 kD, 9 kD, 8 kD, 7 kD, 6 kD, 5 kD, 4 kD, 3 kD, 2 kD or 1 kD. In some examples, small proteins may include proteins that are less than about 50 amino acids, 45 amino acids, 40 amino acids, 35 amino acids or about 30 amino acids. Extremely large or small proteins can be removed by size exclusion chromatography. Extremely large proteins may be isolated by size exclusion chromatography, treated with proteases to produce moderately sized polypeptides and recombined with the moderately size proteins of the sample.

In some cases, proteins may be ordered by size. In some cases, proteins may be ordered by sorting proteins into microwells. In some cases, proteins may be ordered by sorting proteins into nanowells. In some cases, proteins may be ordered by running proteins through a gel such as an SDS-PAGE gel. In some cases, proteins may be ordered by other size-dependent fractionation methods. In some cases, proteins may be separated based on charge, mass, or charge-to-mass ratio. In some cases, proteins may be separated based on hydrophobicity. In some cases, proteins may be separated based on other physical characteristics. In some cases, proteins may be separated under denaturing conditions. In some cases, proteins may be separated under non-denaturing conditions. In some cases, different fractions of fractionated proteins may be placed on different regions of the substrate. In some cases, different portions of separated proteins may be placed on different regions of the substrate. In some cases, a protein sample may be separated in an SDS-PAGE gel and transferred from the SDS-PAGE gel to the substrate such that the proteins are sorted by size in a continuum. In some cases, a protein sample may be sorted into three fractions based on size, and the three fractions may be applied to a first, second, and third region of the substrate, respectively. In some cases, proteins used in the systems and methods described herein may be sorted. In some cases, proteins used in the systems and methods described herein may not be sorted.

Proteins may be tagged, e.g. with identifiable tags, to allow for multiplexing of samples. Some non-limiting examples of identifiable tags include: fluorophores or nucleic acid barcoded base linkers. Fluorophores used may include fluorescent proteins such as GFP, YFP, RFP, eGFP, mCherry, tdtomato, FITC, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Pacific Blue, Coumarin, BODIPY FL, Pacific Green, Oregon Green, Cy3, Cy5, Pacific Orange, TRITC, Texas Red, R-Phycoerythrin, Allophcocyanin, or other fluorophores known in the art.

Any number of protein samples may be multiplexed. For example a multiplexed reaction may contain proteins from 2, 3, 4, 5, 6, 7, 8, 9, 10, 11, 12, 13, 14, 15, 16, 17, 18, 19, about 20, about 25, about 30, about 35, about 40, about 45, about 50, about 55, about 60, about 65, about 70, about 75, about 80, about 85, about 90, about 95, about 100 or more than 100 initial samples. The identifiable tags may provide a way to interrogate each protein as to its sample of origin or may direct proteins from different samples to segregate to different areas on a solid support.

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Protoeforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

### Substrate

In some embodiments, proteins are applied to a functionalized substrate to chemically attach proteins to the substrate. In some cases, the proteins may be attached to the substrate via biotin attachment. In some cases, the proteins may be attached to the substrate via nucleic acid attachment. In some embodiments, the proteins may be applied to an intermediate substance, where the intermediate substance is then attached to the substrate. In some cases, proteins may be conjugated to beads (e.g., gold beads) which may then be captured on a surface (e.g., a thiolated surface). In some cases, one protein may be conjugated to each bead. In some cases, proteins may be conjugated to beads (e.g., one protein per bead) and the beads may be captured on a surface (e.g. in microwells and/or nanowells).

A method of the present disclosure can be carried out at single protein resolution. As used herein, the term “single protein” refers to a protein that is individually manipulated or distinguished from other proteins. A single protein can be a single protein, a single complex of two or more proteins (e.g. a single protein attached to a structured nucleic acid particle or a single protein attached to an affinity agent), or the like. A single protein may be resolved from other proteins based on, for example, spatial or temporal separation from the other proteins. Accordingly, a protein can be detected at “single protein resolution” which is the detection of, or ability to detect, the protein on an individual basis, for example, as distinguished from its nearest neighbor in an array. Reference herein to a ‘single protein’ in the context of a composition, apparatus or method does not necessarily exclude application of the composition, apparatus or method to multiple single proteins that are manipulated or distinguished individually, unless indicated to the contrary.

Alternatively to single protein resolution, a method can be carried out at ensemble-resolution or bulk-resolution. Bulk-resolution configurations acquire a composite signal from a plurality of different proteins or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

The substrate may be any substrate capable of forming a solid support. Substrates, or solid substrates, as used herein can refer to any solid surface to which proteins can be covalently or non-covalently attached. Non-limiting examples of solid substrates include particles, beads, slides, surfaces of elements of devices, membranes, flow cells, wells, chambers, macrofluidic chambers, be flat or curved, or can have other shapes, and can be smooth or textured. In some cases, substrate surfaces may contain microwells. In some cases, substrate surfaces may contain nanowells. In some cases, substrate surfaces may contain one or more microwells in combination with one or more nanowells. In some embodiments, the substrate can be composed of glass, carbohydrates such as dextrans, plastics such as acrylics, polystyrene and copolymers of styrene and other materials, polypropylene, polyethylene, polybutylene, polyurethanes, Teflon™, cyclic olefins, polyimides, nylon, ceramics, resins, Zeonor™, latex, silicon, modified silicon, carbon, metals such as gold, cellulose, inorganic glasses, optical fiber bundles, gels, and polymers and may be further modified to allow or enhance covalent or non-covalent attachment of the oligonucleotides. For example, the substrate surface may be functionalized by modification with specific functional groups, such as maleic or succinic moieties, or derivatized by modification with a chemically reactive group, such as amino, thiol, or acrylate groups, such as by silanization. Suitable silane reagents include aminopropyltrimethoxysilane, aminopropyltriethoxysilane and 4-aminobutyltriethoxysilane. The substrate may be functionalized with N-Hydroxysuccinimide (NETS) functional groups. Glass surfaces can also be derivatized with other reactive groups, such as acrylate or epoxy, using, e.g., epoxysilane, acrylatesilane or acrylamidesilane. The substrate and process for oligonucleotide attachment are preferably stable for repeated binding, washing, imaging and eluting steps. In some examples, the substrate may be a slide or a flow cell.

A method of the present disclosure can be performed in a multiplex format. For example, different proteins can be attached to different addresses in an array. As used herein, the term “array” refers to a population of analytes (e.g. proteins) that are attached to addresses in the array such that the analytes can be distinguished from each other. The term “address,” when used in reference to an array, means a location in the array where a particular analyte (e.g. protein) is present. Proteins in an array can be manipulated or detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding and, optionally non-binding outcomes.

An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

A protein can be attached to an address of an array using any of a variety of means. The attachment can be covalent or non-covalent. Exemplary covalent attachments include chemical linkers such as those achieved using click chemistry or other linkages known in the art or described in US Pat. App. Pub. No. 2021/0101930 A1, which is incorporated herein by reference. Non-covalent attachment can be mediated by receptor-ligand interactions (e.g. (strept)avidin-biotin, antibody-antigen, or complementary nucleic acid strands), for example, in which the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful SNAPs. The use of SNAPs and other moieties to attach proteins to unique identifiers such as tags or addresses in an array are set forth in US Pat. App. Pub. No. 2021/0101930 A1, which is hereby incorporated by reference in its entirety.

An ordered array of functional groups may be created by, for example, photolithography, Dip-Pen nanolithography, nanoimprint lithography, nanosphere lithography, nanoball lithography, nanopillar arrays, nanowire lithography, scanning probe lithography, thermochemical lithography, thermal scanning probe lithography, local oxidation nanolithography, molecular self-assembly, stencil lithography, or electron-beam lithography. Functional groups in an ordered array may be located such that each functional group is less than 200 nanometers (nm), or about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, about 1000 nm, about 1025 nm, about 1050 nm, about 1075 nm, about 1100 nm, about 1125 nm, about 1150 nm, about 1175 nm, about 1200 nm, about 1225 nm, about 1250 nm, about 1275 nm, about 1300 nm, about 1325 nm, about 1350 nm, about 1375 nm, about 1400 nm, about 1425 nm, about 1450 nm, about 1475 nm, about 1500 nm, about 1525 nm, about 1550 nm, about 1575 nm, about 1600 nm, about 1625 nm, about 1650 nm, about 1675 nm, about 1700 nm, about 1725 nm, about 1750 nm, about 1775 nm, about 1800 nm, about 1825 nm, about 1850 nm, about 1875 nm, about 1900 nm, about 1925 nm, about 1950 nm, about 1975 nm, about 2000 nm, or more than 2000 nm from any other functional group. Functional groups in a random spacing may be provided at a concentration such that functional groups are on average at least about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, or more than 100 nm from any other functional group.

The substrate may be indirectly functionalized. For example, the substrate may be PEGylated and a functional group may be applied to all or a subset of the PEG molecules. Additionally, as discussed above, in some cases beads (e.g., gold beads) may be conjugated, and then the beads may be captured on a surface (e.g., a thiolated surface). In some cases, one protein may be conjugated to each bead. In some cases, proteins may be conjugated to beads (e.g., one protein per bead) and the beads may be captured on a surface (e.g. in microwells and/or nanowells).

A solid support or surface may include a plurality of structures or features. A plurality of structures or features may constitute an ordered or patterned array of structures or features. A plurality of structures or features may comprise a non-ordered, non-patterned, or random array of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective center points of neighboring structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. Addresses of an array can have dimensions or pitch that are in the same ranges as those exemplified above for structures or features.

In some cases, a substrate may have a range of different sized microwells such that proteins of different sizes may be sorted into different sized microwells. In some cases, microwells in the substrate may be distributed by size (e.g. with larger microwells distributed in a first region and with smaller microwells distributed in a second region). In some cases, a substrate may have microwells of about ten different sizes. In some cases, a substrate may have microwells of about 20 different sizes, about 25 different sizes, about 30 different sizes, about 35 different sizes, about 40 different sizes, about 45 different sizes, about 50 different sizes, about 55 different sizes, about 60 different sizes, about 65 different sizes, about 70 different sizes, about 75 different sizes, about 80 different sizes, about 85 different sizes, about 90 different sizes, about 95 different sizes, about 100 different sizes, or more than 100 different sizes.

In some cases, a substrate may have nanowells of different sizes. In some cases, nanowells may be about 100 nanometers (nm), about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or between 950 nm and 1 micrometer. In some cases, a substrate may have nanowells that range in size from 100 nm to 1 micrometer. In some cases, a substrate may have nanowells that range in size from 100 nm to 500 nm. In some cases, a substrate may have a range of different sized nanowells such that proteins of different sizes may be sorted into different sized nanowells. In some cases, nanowells in the substrate may be distributed by size (e.g. with larger nanowells distributed in a first region and with smaller nanowells distributed in a second region). In some cases, a substrate may have nanowells of about ten different sizes. In some cases, a substrate may have nanowells of about 20 different sizes, or more than 30 different sizes.

In some cases, a substrate may have a range of different sized nanowells and/or microwells such that proteins of different sizes may be sorted into different sized nanowells and/or microwells. In some cases, nanowells and/or microwells in the substrate may be distributed by size (e.g. with larger microwells distributed in a first region and with smaller nanowells distributed in a second region). In some cases, a substrate may have nanowells and/or microwells of about ten different sizes. In some cases, a substrate may have nanowells and/or microwells of about 20 different sizes, about 25 different sizes, about 30 different sizes, about 35 different sizes, about 40 different sizes, about 45 different sizes, about 50 different sizes, about 55 different sizes, about 60 different sizes, about 65 different sizes, about 70 different sizes, about 75 different sizes, about 80 different sizes, about 85 different sizes, about 90 different sizes, about 95 different sizes, about 100 different sizes, or more than 100 different sizes.

The substrate may comprise any material, including metals, glass, plastics, ceramics or combinations thereof. In some preferred embodiments, the solid substrate can be a flow cell. The flow cell can be composed of a single layer or multiple layers. For example, a flow cell can comprise a base layer (e.g., of borosilicate glass), a channel layer (e.g., of etched silicon) overlaid upon the base layer, and a cover, or top, layer. When the layers are assembled together, enclosed channels can be formed having inlet/outlets at either end through the cover. The thickness of each layer can vary but is preferably less than about 1700 μτη. Layers can be composed of any suitable material known in the art, including but not limited to photosensitive glasses, borosilicate glass, fused silicate, PDMS or silicon. Different layers can be composed of the same material or different materials.

In some embodiments, a method set forth herein can be carried out in a flow cell. For example, a protein array can be housed in a flow cell. Optionally, flow cells can comprise openings for channels that function as ingress and egress, respectively for reagents or fluids set forth herein. In some embodiments, various flow cells of use with embodiments of the present disclosure can comprise different numbers of channels (e.g., 1 channel, 2 or more channels, 3 or more channels, 4 or more channels, 6 or more channels, 8 or more channels, 10 or more channels, 12 or more channels, 16 or more channels, or more than 16 channels). Various flow cells can comprise channels of different depths or widths, which may be different between channels within a single flow cell, or different between channels of different flow cells. A single channel can also vary in depth and/or width. For example, a channel can be less than about 50 microns deep, about 50 microns deep, less than about 100 microns deep, about 100 microns deep, about 100 microns io about 500 microns deep, about 500 microns deep, or more than about 500 microns deep at one or more points within the channel. Channels can have any cross-sectional shape, including but not limited to a circular, a semi-circular, a rectangular, a trapezoidal, a triangular, or an ovoid cross-section.

Proteins may be spotted, dropped, pipetted, flowed, washed or otherwise applied to a substrate. In the case of a substrate that has been functionalized with a moiety such as an NHS ester, no modification of the protein is required. In the case of a substrate that has been functionalized with alternate moieties (e.g. a sulfhydryl, amine, or linker nucleic acid), a crosslinking reagent (e.g. disuccinimidyl suberate, NHS, sulphonamides) may be used. In the case of a substrate that has been functionalized with linker nucleic acid the proteins of the sample may be modified with complementary nucleic acid tags.

In some cases, a protein may be conjugated to a nucleic acid such as a structured nucleic acid particle (SNAP). As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs. Exemplary SNAPs are set forth in U.S. Pat. No. 11,203,612 or U.S. patent application Ser. No. 17/692,035 (granted as U.S. Pat. No. 11,505,796), each of which is hereby incorporated by reference in its entirety.

A protein can be conjugated to a nucleic acid that is a precursor or component of a SNAP. For example, a protein can be attached to a nucleic acid primer and a nucleic acid nanoball may be formed by extension of the primer using a circular nucleic acid template, thereby having the protein linked to the nucleic acid nanoball. When the nucleic acid nanoball is attached to a substrate, the protein attached to the nucleic acid is attached to the substrate by way of the nucleic acid nanoball. A DNA nanoball can be attached (e.g. by adsorption or by conjugation) to a substrate. The substrate may have an amine functionalized surface to which the nucleic acid nanoballs can attach. In some cases, a nucleic acid nanoball may be formed with a functionally active terminus (e.g. a maleimide, NETS-Ester, etc.). The protein may then be conjugated to the nanoball thereby having the protein linked to the nucleic acid nanoball. When the nucleic acid nanoball is attached to a substrate, the protein attached to the nucleic acid is attached to the substrate by way of the nucleic acid nanoball. A DNA nanoball can be attached (e.g. by adsorption or by conjugation) to a substrate. The substrate may have an amine functionalized surface to which the nucleic acid nanoballs can attach. Click chemistry can also be used to attach nucleic acids to a substrate. Similar chemistry can be used to attach other SNAPs to a substrate. Other useful chemistries that can be used to attach proteins to substrates, for example, via intermediate SNAPs are set forth in U.S. Pat. No. 11,203,612 or U.S. patent application Ser. No. 17/692,035 (granted as U.S. Pat. No. 11,505,796), each of which is hereby incorporated by reference in its entirety.

Photo-activatable cross linkers may be used to direct cross linking of a sample to a specific area on the substrate. Photo-activatable cross linkers may be used to allow multiplexing of protein samples by attaching each sample in a known region of the substrate. Photo-activatable cross linkers may allow the specific attachment of proteins which have been successfully tagged, for example by detecting a fluorescent tag before cross linking a protein. Examples of photo-activatable cross linkers include, but are not limited to, N-5-azido-2-nitrobenzoyloxysuccinimide, sulfosuccinimidyl 6-(4′-azido-2′-nitrophenylamino)hexanoate, succinimidyl 4,4′-azipentanoate, sulfosuccinimidyl 4,4′-azipentanoate, succinimidyl 6-(4,4′-azipentanamido)hexanoate, sulfosuccinimidyl 6-(4,4′-azipentanamido)hexanoate, succinimidyl 2-((4,4′-azipentanamido)ethyl)-1,3′-dithiopropionate, and sulfosuccinimidyl 2-((4,4′-azipentanamido)ethyl)-1,3′-dithiopropionate.

Samples may also be multiplexed by restricting the binding of each sample to a discrete area on the substrate. For example, the substrate may be organized into lanes. Another method for multiplexing is to apply the samples iteratively across the substrate, following each sample application with a protein detection step utilizing a nonspecific protein binding reagent or dye. In some cases, examples of dyes may include fluorescent protein gel stains such as SYPRO® Ruby, SYPRO® Orange, SYPRO® Red, SYPRO® Tangerine, and Coomassie™ Fluor Orange.

By tracking the locations of all proteins after each addition of sample it is possible to determine the stage at which each location on the substrate first contained a protein, and thus from which sample that protein was derived. This method may also determine the saturation of the substrate after each application of sample and allows for maximization of protein binding on the substrate. For example, if only 30% of functionalized locations are occupied by protein after a first application of a sample then either a second application of the same sample or an application of a different sample may be made.

Proteins may be attached to a substrate or SNAP by one more amino acid residues. In some examples, the proteins may be attached via one or more of the N terminal, C terminal, both terminals, or an internal residue.

In addition to permanent crosslinkers, it may be appropriate for some applications to use photo-cleavable linkers and that doing so enables proteins to be selectively extracted from the substrate following analysis. In some cases, photo-cleavable cross linkers may be used for several different multiplexed samples. In some cases, photo-cleavable cross linkers may be used from one or more samples within a multiplexed reaction. In some cases, a multiplexed reaction may comprise control samples cross linked to the substrate via permanent crosslinkers and experimental samples cross linked to the substrate via photo-cleavable crosslinkers.

Each conjugated protein may be spatially separated from each other conjugated protein such that each conjugated protein is optically resolvable (e.g. at single molecule resolution). Proteins may thus be individually labeled with a unique spatial address. In some embodiments, this can be accomplished by conjugation using low concentrations of protein and low density of attachment sites on the substrate so that each protein molecule is spatially separated from each other protein molecule. In examples where photo-activatable crosslinkers are used, a light pattern may be used such that proteins are affixed to predetermined locations.

In some methods, bulk proteins that have been purified may be conjugated to a substrate and processed using methods described herein so as to identify the purified protein. Bulk proteins may comprise purified proteins that have been collected together. In some examples, bulk proteins may be conjugated at a location that is spatially separated from each other conjugated protein or bulk proteins such that each conjugated protein or bulk protein is optically resolvable. Proteins, or bulk proteins, may thus be individually labeled with a unique spatial address. In some embodiments, this can be accomplished by conjugation using low concentrations of protein and low density of attachment sites on the substrate so that each protein molecule is spatially separated from each other protein molecule. In examples where photo-activatable crosslinkers are used, a light pattern may be used such that one or more proteins are affixed to predetermined locations.

In some embodiments, each protein may be associated with a unique spatial address. For example, once the proteins are attached to the substrate in spatially separated locations, each protein can be assigned an indexed address, such as by coordinates. In some examples, a grid of pre-assigned unique spatial addresses may be predetermined. In some embodiments the substrate may contain easily identifiable fixed marks such that placement of each protein can be determined relative to the fixed marks of the substrate. In some examples the substrate may have grid lines and/or and “origin” or other fiducials permanently marked on the surface. In some examples the surface of the substrate may be permanently or semi-permanently marked to provide a reference by which to locate cross linked proteins. The shape of the patterning itself, such as the exterior border of the conjugated polypeptides may also be used as fiducials for determining the unique location of each spot.

The substrate may also contain conjugated protein standards and controls. Conjugated protein standards and controls may be peptides or proteins of known sequence which have been conjugated in known locations. In some cases, conjugated protein standards and controls may serve as internal controls in an assay. The proteins may be applied to the substrate from purified protein stocks, or may be synthesized on the substrate through a process such as Nucleic Acid-Programmable Protein Array (NAPPA).

In some examples, the substrate may comprise fluorescent standards. These fluorescent standards may be used to calibrate the intensity of the fluorescent signals from assay to assay. These fluorescent standards may also be used to correlate the intensity of a fluorescent signal with the number of fluorophores present in an area. Fluorescent standards may comprise some or all of the different types of fluorophores used in the assay.

### Affinity Reagents

Once the substrate has been conjugated with the proteins from the sample, affinity reagent measurements can be performed as described below.

An “affinity reagent” is a molecule or other substance that is capable of specifically or reproducibly binding to an analyte (e.g. protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not resulting in a modification to the chemical structure of the analyte to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof. An “unknown affinity reagent” may be any affinity reagent for which binding characteristics toward one or more proteins has not been characterized.

An affinity reagent or other molecule used in a method set forth herein can include a label. As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a luminophore (e.g. fluorophore), chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., sequencing of, or hybridization to, a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “epitope” refers to an affinity target within a protein, polypeptide or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

An affinity reagent can be characterized in terms of its binding affinity. As used herein, the term “binding affinity” or “affinity” refers to the strength or extent of binding between an affinity reagent and a binding partner. In some cases, the binding affinity of an affinity reagent for a binding partner may be vanishingly small or effectively zero. A binding affinity of an affinity reagent for a binding partner may be qualified as being “high affinity,” “medium affinity,” or “low affinity.” A binding affinity of an affinity reagent for a binding partner may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

An affinity reagent, whether known or unknown, may have high, moderate or low specificity for its target. As used herein, the term “promiscuous,” when used in reference to an affinity reagent, means that the affinity reagent is known or suspected to have binding affinity for a variety of different proteins in a given sample. For example, an affinity reagent that is known or suspected to recognize a variety of proteins having different primary sequences is promiscuous. A promiscuous affinity reagent may have high affinity for one or more of the different analytes that it recognizes. A promiscuous reagent may be composed of a single species of reagent, such as a single affinity reagent, or a promiscuous reagent may be composed of two or more different species of reagent. For example, a promiscuous affinity reagent may be composed of a single species of antibody that recognizes a variety of different proteins in a sample, or the promiscuous affinity reagent may be composed of a pool containing several different antibody species that collectively recognize the variety of different proteins in the sample. In other embodiments, an affinity reagent may be non-specific for its target protein.

In some cases, the test affinity reagents (e.g. partially characterized or completely unknown affinity reagent) may be attached to a label. Useful labels include any molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

In some examples, an affinity reagent may bind to amino acid motifs of a given length, such as 2, 3, 4, 5, 6, 7, 8, 9, 10, or more than 10 amino acids. In some examples, an affinity reagent may bind to amino acid motifs of a range of different lengths from 2 amino acids to 40 amino acids.

An affinity reagent (whether unknown or characterized) may bind to naturally occurring or modified amino acid sequences, such as phosphorylated or ubiquitinated amino acid sequences. In some examples, an affinity reagent may bind to two or more different proteins. In some examples, an affinity reagent may bind weakly to its target or targets. For example, the affinity reagent may bind less than 10%, less than 10%, less than 15%, less than 20%, less than 25%, less than 30%, less than 35%, or less than 35% of its target or targets. In some examples, the affinity reagent may bind moderately or strongly to its target or targets. For example, the affinity reagent may bind more than 35%, more than 40%, more than 45%, more than 60%, more than 65%, more than 70%, more than 75%, more than 80%, more than 85%, more than 90%, more than 91%, more than 92%, more than 93%, more than 94%, more than 95%, more than 96%, more than 97%, more than 98%, or more than 99% of its target or targets.

An affinity reagent may be applied at about a 1:1, 2:1, 3:1, 4:1, 5:1, 6:1, 7:1, 8:1, 9:1 or 10:1 excess relative to the proteins that it is suspected to bind. The unknown affinity reagent may be applied at about a 1:1, 2:1, 3:1, 4:1, 5:1, 6:1, 7:1, 8:1, 9:1 or 10:1 excess relative to the expected incidence of the proteins.

### Binding Measurements for Identifying or Characterizing Proteins

The present disclosure provides binding assays that are useful for identifying or characterizing individual unknown proteins among a plurality of unknown proteins (e.g. an array of unknown proteins) using a plurality of previously characterized affinity reagents. The binding assays can be particularly powerful for large pluralities of proteins, for example, pluralities on the scale of a biological proteome. Advantageously, the binding assays can be configured to identify or characterize a number of different proteins that exceeds the number of affinity reagents used. This can be achieved, for example, by (1) using promiscuous affinity reagents that bind to multiple different candidate proteins suspected of being present in a given sample, and (2) subjecting the protein sample to a set of promiscuous affinity agents that, taken as a whole, are expected to bind each candidate protein in a different combination, such that each candidate protein is expected to be encoded by a unique profile of binding and non-binding outcomes. Binding assays can be configured to identify or characterize unknown proteins using characterized affinity reagents as set forth in U.S. Pat. No. 10,473,654; US Pat. App. Pub. Nos. 2020/0318101 A1; 2020/0286584 A1 or 2023/0114905; or Egertson et al., Biokciv (2021), DOI: 10.1101/2021.10.11.463967, each of which is incorporated herein by reference.

Promiscuity of an affinity reagent is a characteristic that can be understood relative to a given population of proteins. Promiscuity can arise due to the affinity agent recognizing an epitope that is known to be present in a plurality of different candidate proteins. For example, epitopes having relatively short amino acid lengths such as dimers, trimers, or tetramers can be expected to occur in a substantial number of different proteins in the human proteome. Alternatively or additionally, a promiscuous affinity reagent can recognize different epitopes (e.g. epitopes differing from each other with regard to amino acid composition or sequence), the different epitopes being present in a plurality of different candidate proteins. For example, a promiscuous affinity agent that is designed or selected for its affinity toward a first trimer epitope may bind to a second epitope that has a different sequence of amino acids when compared to the first epitope.

When endeavoring to characterize or identify unknown proteins in an array, a binding assay can be configured to contact a plurality of different affinity reagents with the array of proteins, wherein the affinity reagents are known or characterized. For example, the degree of promiscuity for the affinity reagents can be known. In this example, each of the affinity reagents can be distinguishable from the other affinity agents, for example, due to unique labeling (e.g. different affinity reagents having different luminophore labels), unique spatial location (e.g. a given affinity reagent being bound at one or more distinguishable addresses in an array), and/or unique time of use (e.g. different affinity reagents being delivered in series to an array of proteins). Accordingly, the plurality of affinity reagents produces a binding profile for each individual protein that can be decoded to identify the individual protein as a particular candidate protein most likely to generate the observed binding profile based on estimates of the probability of each affinity reagent binding to the candidate protein. The individual proteins can be identified based on observed positive binding outcomes alone or in combination with observed negative binding outcomes.

Continuing with the example of a binding assay configured to identify or characterize unknown proteins, distinct and reproducible binding profiles may be observed for one or more of the unknown proteins in a sample. However, in many cases one or more binding events produces inconclusive or even aberrant results and this, in turn, can yield ambiguous binding profiles. For example, observation of binding outcome for a single-molecule binding event can be particularly prone to ambiguities due to stochasticity in the behavior of single molecules when observed using certain detection hardware. The present disclosure provides methods that provide accurate protein identification despite ambiguities and imperfections that can arise in many contexts. In some configurations, methods for identifying, quantitating or otherwise characterizing one or more unknown proteins in a sample utilize a binding model that evaluates the likelihood or probability that one or more candidate proteins that are suspected of being present in the sample will have produced an empirically observed binding profile. The binding model can include information regarding expected binding outcomes (e.g. positive binding outcomes or negative binding outcomes) for binding of one or more affinity reagents with one or more candidate proteins. The information can include an a priori characteristic of a candidate protein, such as presence or absence of a particular epitope in the candidate protein or length of the candidate protein. Alternatively or additionally, the information can include empirically determined characteristics such as propensity or likelihood that the candidate protein will bind, or will not bind to a particular affinity reagent. Accordingly, a binding model can include information regarding the propensity or likelihood of a given candidate protein generating a false positive or false negative binding result in the presence of a particular affinity reagent, and such information can optionally be included for a plurality of affinity reagents.

Methods set forth herein can be used to evaluate the degree of compatibility of one or more empirical binding profiles with results computed for various candidate proteins using a binding model. For example, to identify an unknown protein in a sample of many proteins, an empirical binding profile for the protein can be compared to results computed by the binding model for many or all candidate proteins suspected of being in the sample. In some configurations of the methods set forth herein, identity for the unknown protein is determined based on a likelihood of the unknown protein being a particular candidate protein given the empirical binding pattern or based on the probability of a particular candidate protein generating the empirical binding pattern. Optionally a score can be determined from the measurements that are acquired for the unknown protein with respect to many or all candidate proteins suspected of being in the sample. A digital or binary score that indicates one of two discrete states can be determined. In particular configurations, the score can be non-digital or non-binary. For example, the score can be a value selected from a continuum of values such that an identity is made based on the score being above or below a threshold value. Moreover, a score can be a single value or a collection of values.

### Additional Assays for Identifying or Characterizing Proteins

In some detection assays, a protein can be cyclically modified and the modified products from individual cycles can be detected. In some configurations, an amino acid sequence of a protein can be determined by a sequential process in which each cycle includes steps of detecting the protein and removing one or more terminal amino acids from the protein. Optionally, one or more of the steps can include adding a label to the protein, for example, at the amino terminal amino acid or at the carboxy terminal amino acid. In particular configurations, a method of detecting a protein can include steps of (i) exposing a terminal amino acid on the protein; (ii) detecting a change in signal from the protein; and (iii) identifying the type of amino acid that was removed based on the change detected in step (ii). The terminal amino acid can be exposed, for example, by removal of one or more amino acids from the amino terminus or carboxyl terminus of the protein. Steps (i) through (iii) can be repeated to produce a series of signal changes that is indicative of the sequence for the protein.

In a first configuration of a cyclical protein detection method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. No. 9,625,469 or 10,545,153, each of which is incorporated herein by reference. Methods and apparatus under development by Erisyon, Inc. (Austin, TX) may also be useful for detecting proteins.

In a second configuration of a cyclical protein detection method, a terminal amino acid of a protein can be recognized by an affinity agent that is specific for the terminal amino acid or specific for a label moiety that is present on the terminal amino acid. The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) may also be useful for detecting proteins.

Cyclical removal of terminal amino acids from a protein can be carried out using an Edman-type sequencing reaction in which a phenyl isothiocyanate reacts with a N-terminal amino group under mildly alkaline conditions to form a cyclical phenylthiocarbamoyl Edman complex derivative. The phenyl isothiocyanate may be substituted or unsubstituted with one or more functional groups, linker groups, or linker groups containing functional groups. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. https://doi.org/10.1007/978-1-4899-1031-8_8).

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. Whatever the plexity, the extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

### Binding Measurements for Identifying or Characterizing Test Affinity Reagents

The present disclosure provides methods for identifying or characterizing test affinity reagents (e.g. partially characterized or completely unknown affinity reagents). An affinity reagent can be referred to as a “test affinity reagent” in the context of a given assay when the affinity, specificity, or other binding characteristic of the affinity reagent is to be determined or characterized by the assay. An affinity reagent can be referred to as a “partially characterized or completely unknown affinity reagent” in the context of a given assay when the affinity, specificity, or other binding characteristic of the affinity reagent for one or more proteins in the assay is not known. For example, whether or not a test affinity reagent (e.g. a partially characterized or completely unknown affinity reagent) will demonstrate measurable or detectable binding to one or more protein in the assay may not be known. A test affinity reagent (e.g. partially characterized or completely unknown affinity reagent) can be identified or characterized in a method herein based on observed binding to at least one known or characterized protein. Observed binding can be referred to as a “positive binding outcome.” Moreover, a partially characterized or completely unknown affinity reagent can be identified or characterized based on observed absence of binding to at least one known or characterized protein, referred to as a “negative binding outcome.” The specificity or promiscuity of an affinity reagent can be determined in a method herein based on observations of both positive and negative binding outcomes in a binding assay. A test affinity reagent (e.g. partially characterized or completely unknown affinity reagent) that is observed to selectively bind a particular protein in an array, without substantially binding to other proteins in the array, can be characterized as being specific to that particular protein. A test affinity reagent (e.g. partially characterized or completely unknown affinity reagent) that is observed to bind a particular subset of proteins in an array can be characterized to determine the nature and extent of its promiscuity. The binding assays can be performed as set forth above in the context of characterizing or identifying unknown proteins except that the proteins are known or characterized, and the affinity reagents are partially characterized or completely unknown.

A binding assay can be configured to characterize one or more test affinity reagent (e.g. partially characterized or completely unknown affinity reagents) by contacting the test affinity reagents with an array of different proteins, wherein the identity and location of proteins in the array are known. For example, binding measurements can be used for identifying or characterizing proteins on arrays as set forth above herein. Whether using binding measurements or otherwise, epitopes or amino acid sequences for proteins at respective addresses of the array can be known. In this example, a test affinity reagent (e.g. partially characterized or completely unknown affinity reagent) can be contacted with the array and the addresses where the affinity reagent binds and does not bind can be detected. The binding characteristics of the test affinity reagent can be determined based on the known proteins that are present or absent at each address. More specifically, proteins that are common to addresses that are bound by the test affinity reagent can be identified as target proteins of the test affinity reagent and proteins that are common to addresses that are not observed to bind the test affinity reagent can be identified as non-target proteins for the test affinity reagent.

Continuing with the example of a binding assay configured to identify or characterize a test affinity reagent (e.g. partially characterized or completely unknown affinity reagent), the test affinity reagent may be observed to bind to a distinct and reproducible pattern of addresses in an array of protein addresses. However, in many cases one or more binding and non-binding events produces inconclusive or even aberrant results and this, in turn, can yield ambiguous results. For example, observation of binding outcome for a single-molecule binding event can be particularly prone to ambiguities due to stochasticity in the behavior of single molecules when observed using certain detection hardware. The methods set forth herein for providing accurate protein identification despite ambiguities and imperfections that can arise in the context of single molecule detection, can be used to characterize affinity reagents as well. In some configurations, methods for characterizing a test affinity reagent (e.g. partially characterized or completely unknown affinity reagent) utilizes a binding model that evaluates the likelihood or probability that one or more candidate proteins that are known to be present in a protein array will have produced an empirically observed pattern of protein addresses that bind the affinity reagent. The binding model can include information regarding expected binding outcomes (e.g. positive binding outcomes or negative binding outcomes) for binding of one or more affinity reagents with one or more candidate proteins. The information can include an a priori characteristic of a candidate protein, such as presence or absence of a particular epitope in the candidate protein or length of the candidate protein. Alternatively or additionally, the information can include empirically determined characteristics such as propensity or likelihood that the candidate protein will bind to a particular affinity reagent or a particular type of affinity reagent (e.g. the type can be antibody, aptamer, or other binding reagent composition). Accordingly, a binding model can include information regarding the propensity or likelihood of a given candidate protein generating a false positive or false negative binding result in the presence of a particular affinity reagent.

Each measurement cycle to determine the binding characteristics of a test affinity reagent (e.g. partially characterized or completely unknown affinity reagent) can include several stages. In a first stage, the test affinity reagent can be applied to an array where it may adsorb to the conjugated proteins at one or more address.

Optionally, the substrate can be lightly washed to remove non-specific binding. This washing step can be performed under conditions which will not elute the test affinity reagent which has bound to the immobilized proteins. Some examples of buffers which could be used for this step include phosphate buffered saline, Tris buffered saline, phosphate buffered saline with Tween20, and Tris buffered saline with Tween20.

Following adsorption, the spatial binding addresses on the array where the test affinity reagent bound can be determined, such as through measurement of a fluorophore that has been conjugated to the test affinity reagent directly, or to a complement nucleic acid that hybridizes to a nucleic acid strand conjugated to the test affinity reagent. The detection method can be any that is compatible with the choice of detection moiety. For example, fluorophores and bioluminescent moieties may be optically detected. The unique, spatial address of each immobilized protein from the portion of the proteome located on the substrate may be determined prior to the binding measurements. For example, proteins can be located in an array using a binding assay that is configured to contact a plurality of different affinity reagents with the array of proteins, wherein the affinity reagents are known or characterized. Alternatively, an array of proteins can be produced by attaching known proteins to known addresses. In some embodiments, the fluorescently tagged test affinity reagent (e.g. partially characterized or completely unknown affinity reagent) may be quenched by exposure to prolonged intense light at the activation wavelength. In some embodiments, it may be desirable to cycle n fluorophores to distinguish which signals were derived from the previous n−1 cycles.

### Analysis

Upon acquiring binding data, a step in affinity reagent identification or characterization may comprise systems, instructions, processes, procedures or software which are used to determine the binding characteristics of a test affinity reagent (e.g. partially characterized or completely unknown affinity reagent) based on the identification of the proteins located at each spatial address on the substrate from the information about where the test affinity reagent was bound. For example, a test affinity reagent may be discovered to preferentially bind to proteins containing the tri-peptide HHH. Given this information about the binding characteristics of the test affinity reagent, a database of the proteins in the sample, and list of binding coordinates and pattern of binding and, optionally non-binding, the system may assign a probability that the test affinity reagent binds to the tri-peptide HHH. In one embodiment, the probability may be assigned based on a deconvolution analysis. Here we describe determining the binding probability to a tri-peptide HHH. However, it should be realized that similar methods could be used to determining the binding probability to all 8000 tri-peptides that could occur given the twenty canonical amino acids.

Promiscuity of an affinity reagent is a characteristic that can be understood relative to a given population of proteins. Promiscuity can arise due to the affinity reagent recognizing an epitope that is present in a plurality of different proteins that are known or suspected of being in a sample, such as a human proteome sample. For example, a promiscuous affinity reagent may recognize epitopes having relatively short amino acid lengths such as dimers, trimers, tetramers, pentamers or hexamers, wherein the epitopes are expected to occur in a substantial number of different proteins in a proteome of a human or other species. Alternatively or additionally, a promiscuous affinity reagent can recognize different epitopes (i.e. having a variety of different structures), the different epitopes being present in a plurality of different proteins in a proteome sample. For example, a promiscuous affinity reagent can have a high probability of binding to a primary epitope target and lesser probability for binding to one or more secondary epitope targets, the secondary epitope targets having a different sequence of amino acids when compared to the primary epitope target. Optionally, the secondary epitope targets can be biosimilar to the primary epitope target, for example, in accordance with a BLOSUM62 scoring matrix.

In some embodiments, the identity of one or more proteins that bind a test affinity reagent (e.g. partially characterized or completely unknown affinity reagent) can be determined relative to a threshold degree of accuracy, for example, based on a protein identified within the plurality of proteins. In some embodiments, a method or system may determine the identity of a portion of the plurality of proteins which are bound by a test affinity reagent to a threshold degree of accuracy based on the pattern of binding of the test affinity reagent to the plurality of proteins on an array.

An analysis method or system may utilize a listing of some or all locations in which a test affinity reagent (e.g. partially characterized or completely unknown affinity reagent) did not bind along with the information about the absence of epitopes to determine the protein present. The method or system may also utilize information about which affinity reagents did and did not bind to each address. Thus, the method or system would use the information about both which proteins were present and which proteins were not present in the population of proteins bound and population of proteins unbound by the test affinity reagent.

An analysis method or system may comprise a database. The database may contain sequences of some or all known proteins in the species from which a given sample was obtained. For example, if the sample is known to be of human origin then a database with the sequences of some or all human proteins may be used. If the species of the sample is unknown then a database of some or all protein sequences may be used. The database may also contain the sequences of some or all known protein variants and mutant proteins, and the sequences of some or all possible proteins that could result from DNA frameshift mutations. The database may also contain sequences of possible truncated proteins that may arise from premature stop codons, or from degradation. The database may also contain a variety of different proteoforms.

An analysis method or system may be configured to generate a binding model for an affinity reagent that was previously unknown or not characterized. A binding model of the present disclosure can be configured on an assumption that the characteristics for affinity reagents binding and, optionally non-binding to proteins in a sample, even if the proteins are unknown, can be treated as quantifiable random variables, and that uncertainty about the binding characteristics can be described by probability distributions. Parameters for a plurality of affinity reagents can be determined, for example, based on a binding assay such as one or more of those set forth herein. Optionally, the parameters can also be determined based on a priori knowledge about the affinity reagents (e.g. expected binding affinity for particular proteins).

An advantage of a binding model generated as set forth herein is that it takes into account characteristics of binding and, optionally non-binding reactions that may otherwise adversely affect the accuracy with which proteins can be identified. For example, binding reactions carried out at single-molecule scale (e.g. detecting binding of affinity reagents to proteins that are individually resolved on a protein array) produce stochastic results. Moreover, non-specific binding of affinity reagents, for example, to the surface of an array to which proteins under observation are attached, can also produce errant results. A binding model can be configured to account for stochasticity, non-specific binding, or other factors for improved accuracy when identifying or characterizing proteins.

Optionally, a binding model can include a function for determining probability of a specific binding event occurring between a protein epitope and an affinity reagent. Epitopes evaluated by the model can have any of a variety of characteristics of interest. For example, the epitopes can have a length of at least 1, 2, 3, 4, 5, 6, 10 or more amino acids. Alternatively or additionally, the epitope length can be at most 2, 3, 4, 5 or 6 amino acids. In some cases, the chemical composition of an epitope can be relatively general with regard to chemical characteristics of amino acid side chains (or other moieties) such as charge, polarity, hydropathy, steric size, steric shape or the like. For example, the chemical composition of an epitope can be expressed in terms of biosimilarity to another epitope.

A binding model can be configured to utilize or include a function for calculating a probability of an affinity reagent binding to a plurality of proteins. For example, the function can be configured to calculate a probability of an affinity reagent binding to a particular protein that was present in a binding assay, probabilities of an affinity reagent binding to substantially all the proteins that were present in a binding assay, or a subset of proteins that were present in a binding assay such as at least 25%, 50%, 75%, 90%, 95%, 99% or more of the proteins that were present in a binding assay. Optionally, the function can further consider negative binding outcomes. For example, the probability of an affinity reagent not binding to a protein can be expressed as: P(affinity probe not binding|protein)=1−P(affinity probe binding|protein). In some cases, this approach may be adversely impacted by one or more non-binding events having an outsized impact. For example, an affinity reagent may not bind to a specific site for numerous difficult-to-predict reasons (e.g., protein structure, presence of unexpected post-translational modifications that hinder binding, etc.).

Another approach is to use a blinded uncensored approach in which uncensored decoding is adapted to be more resilient to missed binding events. This can be done by adjusting probabilities for negative binding outcomes. For example, probability of not binding a trimer of unknown identity can be computed for each affinity reagent:

$$θ = {\sum }_{1}^{8000} {p}_{trimer \_ i} * ( 1 - {bp}_{trimer \_ i} )$$

with p_(trimer_i)=probability of the trimer appearing in the proteome (trimer_i frequency)/(total # trimers in proteome).

In some configurations, a binding model can be configured to include or utilize a function for determining probability of a binding event occurring between an affinity reagents. An affinity reagent can be considered as targeting a specific protein to which it binds with particular probability. For example, the probability can be at least 0.01, 0.05, 0.1, 0.25 0.5, 0.75, 0.9, 0.99 or higher. Alternatively, or additionally, the probability can be at most 0.99, 0.9, 0.75, 0.5, 0.25, 0.1, 0.05, 0.01 or lower. The affinity reagent can also be considered to bind one or more secondary proteins with a probability in a range above. The number of additional primary targets can be at least 1, 3, 5, 7, 9, 15, 20 or more proteins (e.g. proteins that are biosimilar to the primary protein). Alternatively, or additionally, the number of additional primary targets can be at most, 20, 15, 9, 7, 5, 3 or 1 proteins. Biosimilar protein targets can be selected based on empirical observations from a binding assay performed with a test affinity reagent (e.g. partially characterized or completely unknown affinity reagent) and/or by computing a pairwise similarity score of a primary protein to every other possible protein of the same length and then selecting one or more of the other proteins with a high similarity score. A similarity score can be computed by summing up similarity between the pair of residues at each sequence location, for example, using BLOSUM62 or other function for determining biosimilarity.

A parameterized binding model can be generated. For example, a binding probability can be assigned to each unique protein recognized by the affinity reagent in a binding assay. Optionally, a non-specific binding rate can be assigned to a binding reagent. The probability of an affinity reagent binding to a given candidate protein can be computed by first computing the probability of a specific binding event happening. The binding model parameters can include a vector of probabilities of the affinity reagent binding to each recognized protein. Furthermore, the model can include a function for computing the probability of a non-specific protein binding event happening. The probability of the affinity reagent binding to the protein and generating a detectable signal can be represented as the probability of one or more specific or non-specific binding events occurring.

In an alternative configuration of systems and methods set forth herein, the probabilities of a negative binding outcome can be calculated by subtracting the probabilities of a positive binding outcome from 1, the probabilities being represented by a value between 0 and 1. Positive and negative binding outcomes can be equally weighted. Alternatively, positive binding outcomes can be weighted more heavily relative to negative binding outcomes. In other cases, negative binding outcomes can be weighted more heavily relative to positive binding outcomes. The latter weighting can be particularly desirable to account for the numerous difficult-to-predict mechanisms by which an affinity reagent may bind to proteins non-specifically.

The results obtained for a test affinity reagent (e.g. partially characterized or completely unknown affinity reagent) can be modeled according to a known or suspected epitope composition and binding results obtained from an assay performed with known proteins. For example, an affinity reagent targeting epitopes of length k (e.g. for a trimer, k=3) can be modeled by assigning a binding probability 0 to each unique target epitope j of length k recognized by the reagent. Further, a protein non-specific binding rate can be assigned p_{nsbepitope} representing the probability of the affinity reagent binding to any epitope in a protein non-specifically. Given the primary sequence for a protein of length M, the probability of an affinity reagent binding to the protein can be computed as follows:

The probability of a specific binding event happening can be expressed by:

$${p}_{specific} = 1 - {\prod }_{j = 1}^{8000} 1 - {θ}_{j}^{{x}_{j}}$$

with:

X: the count of each epitope j in the protein sequence

X={x₁,x₂,x₃. . . x₈₀₀₀} with xⱼ∈ [symbol] *

θ: the binding model parameters. A vector of probabilities of the affinity reagent binding to each recognized epitope

θ={θ₁,θ₂,θ₃, . . . ,θ₈₀₀₀} with 0≤θⱼ≤1

The probability of a non-specific protein binding event happening can be expressed by:

p_{nonspecific}=1−(1−p_{nsbepitope})^{M-k+1}

with:
p_{nsbepitope}: the probability of the affinity reagent non-specifically binding to any epitope in the protein

≤p_{nsbtrimer}≤1
M: the length of the protein sequence
k: the length of the linear epitope(s) recognized by the affinity reagent.

The probability of an affinity reagent binding to a protein and generating a detectable signal can be expressed as the probability of 1 or more specific or non-specific binding events occurring:

p_{proteinbind}=1−(1−p_{specific})*(1−p_{nonspecific})

The probability of binding to each protein can be adjusted to account for additional random surface non-specific binding (NSB). That is, binding of an affinity reagent to an array close enough to a protein address to generate a false-positive binding event. The prevalence of surface NSB can be defined as a probability 0≤p_{surfacensb}≤1 of such a surface NSB event occurring during the acquisition of a single affinity reagent measurement at a single protein location on the array. The adjusted probability of a protein binding event taking into account surface NSB can be:

p_{adjustedbind}=1−(1−p_{proteinbind})*(1−p_{surfacensb})

### Machine Learning

Embodiments may be implemented using software instructions in a method or system which comprises one or more processes, such as a machine learning, deep learning, statistical learning, supervised learning, unsupervised learning, clustering, expectation maximization, maximum likelihood estimation, Bayesian inference, linear regression, logistic regression, binary classification, multinomial classification, or other pattern recognition processes. For example, the system may perform the one or more processes to analyze the information (e.g., as inputs of the one or more process) of (i) the binding characteristic of each affinity reagent, (ii) the database of the proteins in the sample, (iii) the list of binding coordinates, and/or (iv) the pattern of binding and, optionally non-binding of affinity reagents to proteins, in order to generate or assign (e.g., as outputs of the one or more processes) (a) a probable identity to each coordinate and/or (b) a confidence (e.g., confidence level and/or confidence interval) for that identity. Examples of machine learning processes may include support vector machines (SVMs), neural networks, convolutional neural networks (CNNs), deep neural networks, cascading neural networks, k-Nearest Neighbor (k-NN) classification, random forests (RFs), and other types of classification and regression trees (CARTs).

Deep learning is a subset of machine learning. One difference pertaining to neural networks as compared to other machine learning is a depth and complexity of the architecture. This depth and complexity, when properly trained, is how the neural network “learns” complex interactions. As may be appreciated, a neural network may comprise a multitude of layers. Example layers may include convolutional layers, in which volumes of convolutional filters may be applied, and fully-connected (e.g., dense) layers. Each layer may be defined, at least in part, by certain parameters. Example parameters may include biases, weights, and so on. Additionally, a neural network may be defined, at least in part, by certain hyperparameters. Example hyperparameters may include a number of layers, a number of neurons per layer, activation functions, a dropout rate, and so on.

A software-based platform described herein may advantageously leverage one or more neural networks to perform an enhanced gating process. Additionally, the software-based platform may enable the rapid training of new neural networks.

In one embodiment, partial characterization data may be gathered for a set of affinity reagents to help determine their binding characteristics. For example, a set of affinity reagents may be bound to a first protein array to gather binding and non-binding data. The first protein array may have known proteins at each address, so a positive binding by the affinity reagent to the protein at an address would indicate that the affinity reagent had some affinity for the protein at the particular address. However, since many of the affinity reagents bind to multiple target sites, this initial binding data may not be a complete characterization of the affinity reagent. In one embodiment, the binding data from binding affinity reagents to known proteins on the first array may be used to train a neural network to help recognize characteristics of affinity reagents which bind to particular known epitopes on proteins. The trained neural network model of test affinity reagents (e.g. partially characterized affinity reagents) may then be used in a second experiment wherein each affinity reagent is bound to a proteome array where the address of each protein on the array is unknown. By gathering data from the second experiment with unknown proteins on the array and feeding it through the trained neural network, a further characterization of the affinity reagent and its target proteins may be determined.

In another embodiment, a machine learning process is used to determine the proteins which are bound by a particular set of affinity reagents. For example, a proteome array as described herein may be produced and repeatedly bound by a set of affinity reagents having known binding characteristics. In one example the proteome array may be bound in series by a set of 10, 25, 50, 100, 200, 300 or more labeled affinity reagents with known binding characteristics and the data captured from each binding assay. That data may be fed into a neural network to train the neural network to identify characteristics of affinity reagents which bind to certain proteins. A labeled unknown affinity reagent may then be bound to the same proteome array and the data from that experiment fed into the trained neural network to help predict which proteins may be bound by the unknown affinity reagent.

### Calculating Protein Binding by Adaptive Inference

The process set forth below is a way to estimate the binding probability of an affinity reagent for any of the possible 8000 tri-peptides in any target protein. These trimer binding probabilities of an affinity reagent are estimated from a particular collection of protein binding measurements. More specifically, the protein binding measurements are the fractional binding of the affinity reagent to the protein. For example, if 1,000 copies of a single protein are deposited on an array, and after binding, the affinity reagent bound to 250 of the proteins, the fractional binding would be 0.25. The training data sets would have such protein binding measurements for a plurality of different protein sequences.

This process uses a probabilistic model for affinity reagent protein binding built on trimer binding probabilities. With this model, the probability of a single molecule of the affinity reagent binding to a single copy of any protein can be estimated, given the primary sequence of the protein.

The binding probability of an affinity reagent to a protein is modeled as:

$$p = 1 - {\prod }_{j = 1}^{8000} 1 - {θ}_{j}^{{x}_{j}}$$

with:
X: the count of each trimer in the protein bound to a substrate

X={x₁,x₂,x₃. . . x₈₀₀₀} with xⱼ∈ [symbol] *
θ: the binding model parameters. A vector of probabilities of the affinity reagent not binding each trimer

θ={θ₁,θ₂,θ₃, . . . } with 0≤θⱼ≤1

The probability of an affinity reagent not binding to a protein is equal to 1−p.

The likelihood of a particular affinity reagent binding model given an observed binding outcome to a single protein substrate is:

$$L ( θ \vert y , X ) = {( {\prod }_{j = 1}^{8000} {θ}_{j}^{{x}_{j}} )}^{1 - y} {( 1 - {\prod }_{j = 1}^{8000} 1 - {θ}_{j}^{{x}_{j}} )}^{y}$$

with:
y: the binding outcome

$$y = \{ \begin{matrix}1 & if binding \\ 0 & if no binding\end{matrix}$$

The log-likelihood is:

$$log L ( θ \vert y , X ) = ( 1 - y ) {\sum }_{j = 1}^{8000} {x}_{j} log {θ}_{j} + y log 1 - {\prod }_{j = 1}^{8000} {θ}_{j}^{{x}_{j}}$$

The log likelihood of multiple binding outcomes may also be calculated for the affinity reagents.

If N binding outcomes are observed for N single-copy proteins:

Y is a vector of N binding outcomes

X is an N×8000 matrix of trimer counts for each of the N substrates where any row X, is the counts for each of 8000 trimers for the i th substrate.

The log likelihood of a binding model θ given this set of observations is:

$$log L ( θ \vert Y , X ) = {\sum }_{i = 1}^{N} ( ( 1 - {y}_{i} ) {\sum }_{j = 1}^{8000} {x}_{i j} log {θ}_{j} + {y}_{i} log ( 1 - {\prod }_{j = 1}^{8000} {θ}_{j}^{{x}_{i j}} ) )$$

In the process, the maximum of the above likelihood function may provide an estimate of the parameters of the affinity reagent binding model. With the current formulation, multiple binding outcomes to copies of the same protein would be represented as individual entries in the vector, and rows in the matrix which may lead to a large memory footprint when using software to compute the log likelihood. To reduce the computational resources required, the same calculation may be performed using a more compact representation of the binding data where binding outcomes are collapsed into the number of binding and non-binding events for each unique protein substrate in the collection of outcomes. With this reformulation, the dimensions of the matrices and vectors used for the computation scales with the number of unique proteins in the data set rather than the number of binding outcomes observed.

In an alternative formulation:

$$log ( L ( θ \vert U , B , T ) ) = {\sum }_{i = 1}^{M} ( {u}_{i} {\sum }_{j = 1}^{8000} {t}_{i j} log {θ}_{j} + {b}_{i} log ( 1 - {\prod }_{j = 1}^{8000} {θ}_{j}^{{t}_{i j}} ) )$$

with:
binding observations consisting of M unique proteins
B is a vector of binding counts and U is a vector of non-bind counts to each unique protein. Both of length M
T is the matrix of trimer counts for each unique protein with M rows and 8000 columns.

the process estimates the binding model \hat{θ} by minimizing the function ƒ(\hat{θ})=−log (L(\hat{θ}|U, B, T)) using the L-BFGS-B algorithm with each parameter of θ constrained to be greater than zero and less than one.

The speed of a maximum likelihood estimation is improved by providing a function J=j(\hat{θ}) to directly compute the analytical gradient vector J off given a parameter estimate B. The analytical gradient is:

$$J = j ( \hat{θ} ) = [ \frac{\partial f}{\partial {\hat{θ}}_{1}} \frac{\partial f}{\partial {\hat{θ}}_{2}} … … … … \frac{\partial f}{\partial {\hat{θ}}_{8000}} ]$$

$$\frac{\partial f}{\partial {\hat{θ}}_{q}} = - {\sum }_{i = 1}^{M} \frac{{u}_{i} {t}_{i q}}{{\hat{θ}}_{q}} - \frac{{b}_{i} {t}_{i q} {Π}_{j = 1}^{8000} {\hat{θ}}_{j}^{{t}_{ij}}}{{\hat{θ}}_{q} ( 1 - {Π}_{j = 1}^{8000} {\hat{θ}}_{j}^{{t}_{ij}} )}$$

As demonstrated, the above approach works with individual counts of binding and non-binding events for each unique protein in the set of experimental observations. However, in cases where only fractional binding can be determined or estimated, the vector can be replaced with a vector having a length containing the fractional binding to each of the unique proteins in the data set and the vector replaced with the vector. For example, in the case of binding a fluorescently-labeled affinity reagent candidate to a nucleic acid programmable protein array (NAPPA) followed by imaging to measure fluorescence intensity, values for may be estimated as the fluorescence intensity for a given protein divided by an estimate of the fluorescence intensity that would be observed at a fractional binding of 1 (all protein bound). In cases where a particular trimer is not observed at all in the binding data set, only observed in a single unique protein sequence, or if the set of proteins containing a particular trimer is the exact same set as another trimer, the binding probability of the trimer may be difficult to determine. For such trimers, the binding probability may be imputed, for example by setting the probability of the trimer to be equivalent to that of another similar trimer based on amino acid sequence or biochemical characteristics of the trimer. Imputation can be performed by setting the probability of the trimer to the average binding probability of all other trimers that were not imputed.

The above approach may be applied to learn binding models built from dimers, trimers, 4-mers, 5-mers, etc. Furthermore, it may also be applied to models comprising subsets or mixtures of the aforementioned -mers. To do so, the columns representing the counts in each protein for each trimer (1-8000), would be replaced with columns representing the counts of each n-mer in each protein in the matrix M. Further, the parameter vector would be the binding probability to each of the n-mers used in the model.

### Determining Characteristics of Test Affinity Reagent

Once decoding is complete, the probable identities of the proteins conjugated to each spatial address are defined and their abundance in the mixture can be estimated by counting observations. Thus, the binding characteristics and identification of the test affinity reagent (e.g. partially characterized or completely unknown affinity reagent) is determined.

### Profiling Affinity Reagents

A method of the present disclosure can be configured to profile an affinity reagent with respect to one or more characteristics. In some configurations, the method can include steps of (a) providing a substrate having a plurality of attached proteins corresponding to at least a portion of a proteome, wherein each attached protein has a unique spatial address on the substrate, wherein the identity of the protein at each said spatial address is unknown; (b) determining the identity of the protein at each spatial address; (c) testing affinity reagents from a sample by (i) applying an affinity reagent from the sample to the substrate under a first condition, (ii) determining the one or more spatial addresses where the affinity reagent binds under the first condition, and optionally determining one or more spatial addresses where the affinity reagent does not bind, and (iii) repeating (i) and (ii) under a second condition instead of the first condition, the second condition differing from the first condition, wherein the affinity reagent tested under the first condition has identical composition to the affinity reagent tested under the second condition; and (d) determining a binding characteristic of the affinity reagent based on the testing of the affinity reagents from the sample. Optionally, (iii) can include repeating (i) and (ii) a plurality of times, each time under a different condition, wherein the affinity reagent tested under the different conditions has identical composition to the affinity reagent tested under the first condition.

A method of profiling an affinity reagent can use any of a variety of techniques for determining the identity of proteins at spatial addresses including, but not limited to the binding measurements or protein sequencing methods set forth herein. For example, step (b) in the above method can include (1) applying a set of known affinity reagents to the substrate and measuring whether the known set of affinity reagents binds, or does not bind to the attached proteins and (2) identifying the proteins according to the machine learning model. Optionally, step (d) of the method can be carried out by inputting the binding characteristics of the affinity reagents from the sample to the trained machine learning model, thereby determining the binding characteristic of the affinity reagent.

A protein array can be assayed to identify proteins before or after performing profiling or characterizing steps set forth herein. Generally, an array that is used to characterize or profile an affinity reagent will contain known proteins. The identity of the proteins at some or all addresses may be known prior to performing profiling or characterizing steps set forth herein. For example, step (b) can be carried out before step (c) in the above method. However, the identities of the proteins at given addresses need not be known prior to performing profiling or characterizing steps set forth herein. Instead, the array can be assayed to identify proteins after profiling and characterizing an affinity reagent. For example, step (b) can be carried out after step (c) in the above method. Some techniques used to identify proteins on an array may be destructive to the proteins including, for example, amino acid sequencing techniques. In such configurations, proteins on the array can be identified after profiling affinity reagents. In some cases, proteins on the array are denatured during identification steps, for example, it may be beneficial to denature the proteins to facilitate binding to known affinity reagents. Although it may be possible or even desirable to profile affinity reagents using the denatured proteins, in some cases native state proteins may be required or preferred when profiling affinity reagents. Thus, it may be beneficial to identify proteins at respective addresses after profiling affinity reagents.

An affinity reagent can be profiled to determine any of a variety of characteristics. Exemplary characteristics include, but are not limited to, concentration dependence of affinity reagent binding; identity of competitors or inhibitors of affinity reagent binding to one or more proteins; concentration dependence of competitors or inhibitors of affinity reagent binding to one or more proteins; dependence of affinity reagent binding on conditions such as pH, ionic strength, redox state, solvent polarity or temperature; effect of various substances on affinity reagent activity such as detergents, salts, buffers or adjuvant compositions; or kinetics of affinity reagent binding to one or more proteins. An affinity reagent can be evaluated with respect to characteristics that are pertinent to a particular use. For example, an affinity reagent can be evaluated with respect to characteristics for use in a diagnostic, prognostic or research test. In another example, an affinity reagent can be evaluated with respect to characteristics for use as a therapeutic agent.

An affinity reagent that is profiled using a method of the present disclosure can be labeled to facilitate detection. Any of a variety of labels known in the art or set forth herein can be used.

A method of profiling an affinity reagent can be configured to determine concentration dependence of affinity reagent binding to various proteins in an array. For example, an array having known proteins can be contacted with various amounts or concentrations of an affinity reagent, and the binding results can be evaluated to determine a binding characteristic for the affinity reagent such as the half maximal effective concentration (EC50), equilibrium dissociation constant (K_{D}) or equilibrium association constant (K_{A}). Such methods can be used to determine concentration dependence of an affinity reagent for binding to at least 1, 2, 3, 4, 5, 10, 25, 50, 100 or more different proteins in an array. The concentration dependence can differ for different proteins in the array.

A method of profiling an affinity reagent can be configured to identify or characterize competitors or inhibitors. For example, an array having known proteins can be contacted with an affinity reagent in the presence of other substances to evaluate the ability of the substances to function as competitors for proteins that bind to the affinity reagent or to evaluate the ability of the substances to function as inhibitors of proteins that bind to the affinity reagent. For example, a fluid that is in contact with an array of known proteins can include the affinity reagent that is to be tested and can also include a peptide or protein having an epitope that is known or suspected of being present in at least one protein in the array. The epitope can be a linear amino acid epitope (e.g. a trimer, tetramer, pentamer, hexamer etc.) or a conformational epitope. Such methods can be used to identify a competitor or inhibitor for binding of an affinity reagent to at least 1, 2, 3, 4, 5, 10, 25, 50, 100 or more different proteins in an array. The different proteins can compete with, or be inhibited by, the same substance or different substances. The extent of competition or inhibition for a substance can be determined by titrating the amount of the substance that produces an inhibitory or competitive effect. Accordingly, an equilibrium inhibition constant (Ki) can be determined.

A method of profiling an affinity reagent can be configured to identify or characterize one or more conditions that alter binding of the affinity reagent to one or more proteins in an array when the condition(s) is varied. Exemplary conditions that can be varied include, but are not limited to, pH, ionic strength, redox state, solvent polarity or temperature. For example, an array having known proteins can be contacted with an affinity reagent in fluids having varying ranges of a given condition to evaluate the ability of the conditions to increase or decrease binding of the affinity reagent to one or more proteins in the array. For example, a series of fluids that are in contact with an array of known proteins can vary with regard to pH, ionic strength, redox state, solvent polarity or temperature. Such methods can be used to identify conditions that can be varied to increase or decrease binding of an affinity reagent to at least 1, 2, 3, 4, 5, 10, 25, 50, 100 or more different proteins in an array.

A method of profiling an affinity reagent can be configured to determine kinetics of affinity reagent binding to various proteins in an array. For example, an array having known proteins can be contacted with affinity reagents and binding detected after varying durations. In some cases, the amounts or concentrations of the affinity reagents can also be varied. The results can be evaluated to determine a kinetic characteristic for the affinity reagents such as the association rate (kₒₙ) or dissociation rate (k_{off}). Such methods can be used to determine kinetic characteristics of an affinity reagent for binding to at least 1, 2, 3, 4, 5, 10, 25, 50, 100 or more different proteins in an array. The kinetic characteristics can differ for different proteins in the array.

### Characterizing Protein Reactants or Ligands

The present disclosure provides methods for characterizing a protein ligand or protein reactant. In some cases, the protein ligand or reactant is a therapeutic agent or candidate therapeutic agent. A therapeutic agent or candidate therapeutic agent can be categorized, for example, as an enzyme inhibitor (e.g. kinase inhibitor, phosphatase inhibitor, G-protein coupled receptor inhibitor etc.) analgesic, antacid, anxiolytic, sedative, tranquilizer, antiarythmic, antibacterial, antibiotic, anticoagulant, anticonvulsant, antidepressant, antidiarrheal, antiemetic, antifungal, antihistamine, antihypertensive, anti-inflammatory, antineoplastic, antipsychotic, antipyretic, antiviral, barbiturate, beta-blocker, bronchodilator, decongestant, steroid, corticosteroid, expectorant, cough suppressant, mucolytic, cytotoxin, decongestant, diuretic, hormone, hypoglycemic, immunosuppressant, laxative, muscle relaxant, serotonin-reuptake inhibitor, or vitamin. Optionally, a therapeutic agent or candidate can target cancer, examples of which include monoclonal antibodies; growth inhibitors, such as tyrosine kinase inhibitors, proteosome inhibitors, mTOR inhibitors, p13K inhibitors, histone deacetylase inhibitors, hedgehog pathway blockers, BRAF inhibitors or MEK inhibitors; antiangiogenics or PARP inhibitors. A protein ligand or reactant used in a method set forth herein need not be a therapeutic agent, for example, being non-therapeutic or being in a category that differs from one or more of the foregoing categories.

Optionally, a protein ligand or reactant is known or suspected to target a particular type of protein such as a receptor or enzyme. A receptor can be a membrane receptor (e.g. transmembrane receptor) or cytosolic receptor. An enzyme can be a hydrolase, oxidoreductase, lyase, transferase, ligase or isomerase. A protein ligand or reactant can bind or modify a kinase, phosphatase, polymerase, transcription factor, ribosome, protease, metabolic enzyme, G-protein coupled receptor, ion channel, nuclear receptor, hormone receptor, integrin, cytochrome P450, receptor tyrosine kinases, JAK-STAT receptor, receptor serine-threonine kinase, Toll-like receptor or TNF-α receptor.

Exemplary protein ligands and reactants include, but are not limited to, small molecules (smaller than 1500 Daltons), natural products, synthetic products (e.g. from combinatorial libraries), proteins, nucleic acids (e.g. RNA, DNA, mRNA, RNAi, small RNA, antisense RNA) lipids, saccharides, glycans, amino acids, or nucleotides. A protein ligand or reactant can be labeled to facilitate detection. Any of a variety of labels known in the art or set forth herein can be used. Optionally, a protein ligand or reactant can include a nucleic acid tag and the tag can be used as a handle for attaching a label. For example, the label can be attached to a nucleic acid strand that is complementary to a nucleotide sequence in the tag. Particularly useful nucleic acid tags include those found in products of combinatorial synthesis. Labeled oligonucleotides can be hybridized to regions of the tag sequence that are common to members of the library.

A method of characterizing a protein ligand can include steps of (a) providing a substrate having a plurality of attached proteins corresponding to at least a portion of a proteome, wherein each attached protein has a unique spatial address on the substrate, wherein the identity of the protein at each said spatial address is unknown; (b) determining the identity of the protein at each spatial address; (c) applying a ligand to the substrate; (d) determining one or more spatial addresses where the ligand binds, and optionally, determining one or more spatial addresses where the ligand does not bind; and (e) identifying at least one protein on the array to which the ligand binds.

A method of characterizing a protein reactant can include steps of (a) providing a substrate having a plurality of attached proteins corresponding to at least a portion of a proteome, wherein each attached protein has a unique spatial address on the substrate, wherein the identity of the protein at each said spatial address is unknown; (b) determining the identity of the protein at each spatial address; (c) applying a reactant to the substrate; (d) determining one or more spatial addresses having a protein that is modified by the reactant, and optionally determining one or more spatial addresses having a protein that is not modified by the reactant; and (e) identifying at least one protein on the array that is modified by the reactant.

A method of characterizing a protein reactant can use any of a variety of techniques for determining the identity of proteins at spatial addresses including, but not limited to the binding measurements and protein sequencing methods set forth herein. For example, step (b) in the above method can include (1) applying a set of known affinity reagents to the substrate and measuring whether the known set of affinity reagents binds, or does not bind to the attached proteins and (2) identifying the proteins according to the machine learning model. Optionally, step (d) of the method can be carried out by inputting the binding characteristics of the affinity reagents from the sample to the trained machine learning model, thereby determining the binding characteristic of the affinity reagent.

A protein array can be assayed to identify proteins before or after using the array to characterize a protein ligand or protein reactant. Generally, an array that is used to characterize a protein ligand or reactant will contain known proteins. The identity of the proteins at some or all addresses of an array may be known prior to using the array to characterize a protein ligand or reactant. For example, step (b) can be carried out before steps (c) and (d) in the above method. However, the identities of the proteins at given addresses need not be known prior to characterizing a protein ligand or reactant. Instead, the array can be assayed to identify proteins after characterizing a protein ligand or reactant. For example, step (b) can be carried out after steps (c) and (d) in the above method. Some techniques used to identify proteins on an array may be destructive to the proteins including, for example, amino acid sequencing techniques. In such configurations, proteins on the array can be identified after characterizing a protein ligand or reactant. In some cases, proteins on the array are denatured during identification steps. For example, it may be beneficial to denature the proteins to facilitate binding to known affinity reagents. Although it may be possible or even desirable to characterize a protein ligand or reactant using the denatured proteins, in some cases native state proteins may be required or preferred when characterizing a protein ligand or reactant. Thus, it may be beneficial to identify proteins at respective addresses after characterizing a protein ligand or reactant.

A protein, for example, at an address in an array, can optionally be detected based on its enzymatic or biological activity. For example, a protein can be contacted with a reactant that is converted to a detectable product by an enzymatic activity of the protein. Optionally, a protein having a known enzymatic function can be contacted with another substance such as a ligand, reactant, inhibitor, or competitor to determine if the enzymatic function of the protein is changed. Exemplary changes that can be observed include, but are not limited to, activation of the enzymatic function, inhibition of the enzymatic function, attenuation of the enzymatic function, degradation of the protein or competition for a reactant or cofactor used by the protein. Proteins can also be detected based on their binding interactions with other molecules such as affinity reagents, ligands, or reactants. For example, a protein that participates in a signal transduction pathway can be characterized by detecting binding to a second protein that is known to be a binding partner for the candidate protein in the pathway.

### Computer Control Systems

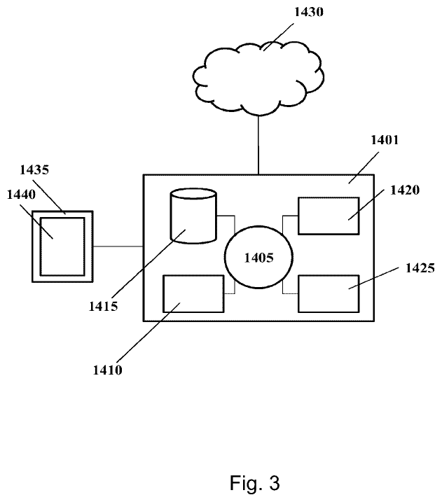

The present disclosure provides computer control systems that are programmed to implement methods of the disclosure. FIG. 3 shows a computer system 1401 that is programmed or otherwise configured to characterize and identify biopolymers, such as proteins. The computer system 1401 can regulate various aspects of assessing and analyzing samples of the present disclosure, such as, for example, observing signals at unique spatial addresses of a substrate; determining a presence of an identifiable tag linked to a biopolymer portion at unique spatial addresses based on observed signals; assessing the determined identifiable tags against a database of biopolymer sequences to determine characteristics of biopolymer portions. The computer system 1401 can be an electronic device of a user or a computer system that is remotely located with respect to the electronic device. The electronic device can be a mobile electronic device.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm can, for example, determine characteristics and/or identities of biopolymer portions, such as protein portions. For example, algorithms may be used to determine a most likely identity of a candidate biopolymer portion, such as a candidate protein portion.

In some embodiments aptamers or peptamers which recognize short epitopes present in many different proteins may be referred to as digital aptamers or digital peptamers. The present disclosure provides a set of digital aptamers or digital peptamers, wherein the set comprises at least about 15 digital aptamers or digital peptamers, wherein each of the 15 digital aptamers or digital peptamers has been characterized to bind specifically to a different epitope consisting of 3 or 4 or 5 consecutive amino acids, and wherein each digital aptamer or digital peptamer recognizes a plurality of distinct and different proteins that comprise the same epitope to which the digital aptamer or digital peptamer binds. In some embodiments the set of digital aptamers or digital peptamers comprises 100 digital aptamers or digital peptamers that bind epitopes consisting of 3 consecutive amino acids. In some embodiments the set of digital aptamers or digital peptamers further comprises 100 digital aptamers that bind epitopes consisting of 4 consecutive amino acids. In some embodiments the set of digital aptamers or digital peptamers further comprises 100 digital aptamers or digital peptamers that bind epitopes consisting of 5 consecutive amino acids. In some cases, digital affinity reagents may be an antibody, aptamer, peptamer, peptide or Fab fragment.

In some embodiments the set of digital aptamers comprises at least about 20, 30, 40, 50, 60, 70, 80, 90, 100, 200, 300, 400, 500, 600, 700 800, 900, or 1000 digital aptamers. In some embodiments the set of digital aptamers comprises at least 1000 digital aptamers that bind epitopes consisting of 4 consecutive amino acids. In some embodiments the set of digital aptamers further comprises at least 100 digital aptamers that bind epitopes consisting of 5 consecutive amino acids. The set of digital aptamers further comprises at least 100 digital aptamers that bind epitopes consisting of 3 consecutive amino acids. In some embodiments the set of digital aptamers are immobilized on a surface. In some embodiments the surface is an array.

In another aspect the present disclosure provides a method for generating a protein binding profile of a sample comprising a plurality of different proteins, said method comprising: contacting said sample with a set of digital aptamers, under conditions that permit binding, wherein the set of digital aptamers comprises at least about 15 digital aptamers, wherein each of the 15 digital aptamers has been characterized to bind specifically to a different epitope consisting of 3 or 4 or 5 consecutive amino acids, and each digital aptamer recognizes a plurality of distinct and different proteins that comprise the same epitope to which the digital aptamer binds; optionally removing an unbound protein; and detecting binding and non-binding of protein to said digital aptamers, whereby a protein binding profile of the sample is generated.

In some embodiments the method further comprises the step of treating the sample with a protein cleaving agent prior to step (a) of contacting the sample with the set of digital aptamers under conditions that permit binding.

In another aspect the present disclosure comprises a library of protein binding profiles for two or more different samples each of which comprises a plurality of proteins, said method comprising: contacting a sample with a set of digital aptamers under conditions that permit binding, wherein the set of digital aptamers comprises at least about 15 digital aptamers, wherein each of the 15 digital aptamers has been characterized to bind specifically to a different epitope consisting of 3 or 4 or 5 consecutive amino acids, and each digital aptamer recognizes a plurality of distinct and different proteins that comprise the same epitope to which the digital aptamer binds; optionally removing an unbound protein; generating a protein binding profile of the sample being tested by detecting binding and non-binding of protein to the digital aptamers, whereby a protein binding profile is generated; and repeating the steps above with at least two samples.

In some embodiments the method further comprises the step of treating the sample with a protein cleaving agent prior to the step of contacting the sample with the set of digital aptamers under conditions that permit binding.

In another aspect the present disclosure comprises a method for characterizing a test sample, comprising: contacting the test sample with a set of digital aptamers under conditions that permit binding, wherein the set of digital aptamers comprises at least about 15 digital aptamers, wherein each of the 15 digital aptamers has been characterized to bind specifically to a different epitope consisting of 3 or 4 or 5 consecutive amino acids, and each digital aptamer recognizes a plurality of distinct and different proteins that comprise the same epitope to which the digital aptamer binds; optionally removing an unbound protein generating a protein binding profile of said test sample by detecting binding and non-binding of protein to the digital aptamers; and comparing the generated protein binding profile of the test sample with a protein binding profile of a reference sample to characterize the test sample.

In another aspect the present disclosure comprises a method for determining presence or absence of a bacteria, virus, or cell in a test sample, said method comprising: contacting the test sample with a set of digital aptamers under conditions that permit binding, wherein the set of digital aptamers comprises at least about 15 digital aptamers, wherein each of the 15 digital aptamers has been characterized to bind specifically to a different epitope consisting of 3 or 4 or 5 consecutive amino acids, and each digital aptamer recognizes a plurality of distinct and different proteins that comprise the same epitope to which the digital aptamer binds; optionally removing an unbound protein; generating a protein binding profile of the test sample by detecting binding and non-binding of protein to the digital aptamers, whereby a protein binding profile is generated; and comparing the protein binding profile of the test sample with a protein binding profile of a reference sample, whereby presence or absence of the bacteria, virus or cell in the test sample is determined by the comparison.

In another aspect the present disclosure comprises a method for identifying or characterizing a test protein in a sample, said method comprising: contacting a sample comprising or suspected of comprising the test protein with a set of digital aptamers that comprises at least about 15 digital aptamers, wherein each of the 15 digital aptamers has been characterized to bind specifically to a different epitope consisting of 3 or 4 or 5 consecutive amino acids, and each digital aptamer recognizes a plurality of distinct and different proteins that comprise the same epitope to which the digital aptamer binds; and determining the identity of the test protein by detecting of binding and non-binding of the test protein to the set of digital aptamers, wherein at least about six digital aptamers bind the test protein; and wherein presence of binding indicates presence of at least about six epitopes in the test protein, wherein the identity of the at least about six epitopes is used to identify the test protein.

While embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of more fully characterizing a partially characterized affinity reagent using a machine learning model, the method comprising:
providing a substrate having a plurality of attached proteins corresponding to at least a portion of a proteome, wherein each attached protein has a unique spatial address on the substrate, wherein the identity of the protein at each said spatial address is unknown;
determining the identity of the proteins at each spatial address by 1) applying a set of known affinity reagents to the substrate and measuring whether the known set of affinity reagents binds, or does not bind to the attached proteins and 2) identifying the proteins according to the machine learning model;
applying a partially characterized affinity reagent to the substrate;
determining the one or more spatial addresses where the partially characterized affinity reagent binds, and does not bind, to the substrate; and
inputting the binding characteristics of the partially characterized affinity reagent to the trained machine learning model to more fully characterize the partially characterized affinity reagent.

2. The method of claim 1, wherein determining the one or more spatial addresses where the partially characterized affinity reagent binds, and does not bind, comprises observing one or more signals produced by the partially characterized affinity reagent when bound to the spatial addresses on the substrate.

3. The method of claim 1, wherein the affinity reagent recognizes a specific epitope that is present in more than one of the plurality of attached proteins.

4. The method of claim 3, wherein the partially characterized affinity reagent is an antibody which binds to a particular trimer amino acid sequence.

5. The method of claim 1, wherein providing a substrate having a plurality of attached proteins corresponding to at least a portion of a proteome comprises providing a substrate having a plurality of proteins corresponding to at least 90% of the proteome of a cell or tissue.

6. The method of claim 1, wherein more fully characterizing the partially characterized affinity reagent comprises determining a probability that the partially characterized affinity reagent will bind to a particular target protein.

7. The method of claim 1, wherein the partially characterized affinity reagent is modified from its endogenous form to be conjugated to an identifiable tag.

8. The method of claim 7, wherein the identifiable tag is a fluorescent tag.

9. The method of claim 7, wherein the identifiable tag is a nucleic acid barcode.

10. The method of claim 1, wherein the machine learning model comprises a function for determining a probability of a positive binding outcome occurring between the partially characterized affinity reagent and at least one protein of the plurality of attached proteins.

11. The method of claim 10, wherein the machine learning model comprises a function for determining the probability of a negative binding outcome occurring between the partially characterized affinity reagent and at least one protein of the plurality of attached proteins.

12. The method of claim 11, wherein the machine learning model weighs the positive binding outcome more heavily relative to the negative binding outcome.

13. The method of claim 11, wherein the machine learning model weighs the negative binding outcome equal to the positive binding outcome.

14. The method of claim 10, wherein the machine learning model comprises a function for determining probability of a specific binding event occurring between the partially characterized affinity reagent and one or more proteins on the substrate.

15. The method of claim 10 wherein the machine learning model comprises a function for determining probability of a non-specific binding event occurring between the partially characterized affinity reagent and one or more proteins on the substrate.

16. A system for more fully characterizing a partially characterized affinity reagent using a machine learning model, comprising:
a substrate having a plurality of proteins corresponding to a portion of a proteome bound thereto, wherein one or more binding sites for each bound protein has been identified and each bound protein has a unique, optically resolvable, spatial address on the substrate; and
a processor configured to execute instructions that when run on the processor perform the method of:
determining the identity of the proteins at each spatial address by 1) applying a set of known affinity reagents to the substrate and measuring whether the known set of affinity reagents binds, or does not bind to the attached proteins and 2) identifying the proteins according to the machine learning model;
applying a partially characterized affinity reagent to the substrate;
determining the one or more spatial addresses where the partially characterized affinity reagent binds, and does not bind, to the substrate; and
inputting the binding characteristics of the partially characterized affinity reagent to the trained machine learning model to more fully characterize the partially characterized affinity reagent.

17. A method of identifying binding characteristics of a partially characterized or completely unknown affinity reagent using a machine learning model, the method comprising:
providing a substrate having a plurality of attached proteins, wherein the identity of the proteins at each position has been determined by identifying the binding of a known set of affinity reagents to the substrate and the binding characteristics of each affinity reagent were calculated according to the machine learning model;
applying a partially characterized or completely unknown affinity reagent to the substrate; and
inputting the binding characteristics of the partially characterized or completely unknown affinity reagent to the machine learning model to identify the binding characteristics of the partially characterized or completely unknown affinity reagent.

18. A method of profiling a test affinity reagent, comprising
(a) providing a substrate comprising a plurality of attached proteins corresponding to at least a portion of a proteome, wherein each attached protein comprises a unique spatial address on the substrate, wherein the identity of the protein at each said spatial address is unknown;
(b) determining the identity of the protein at each spatial address;
(c) testing affinity reagents from a sample by applying a test affinity reagent from the sample to the substrate under a first condition,
(ii) determining the one or more spatial addresses where the test affinity reagent binds under the first condition, and optionally determining one or more spatial addresses where the test affinity reagent does not bind, and
(iii) repeating (i) and (ii) under a second condition instead of the first condition, the second condition differing from the first condition, wherein the test affinity reagent tested under the first condition has identical composition to the test affinity reagent tested under the second condition; and
(d) determining a binding characteristic of the test affinity reagent based on the testing of the affinity reagents from the sample.

19. A method of characterizing a protein ligand, comprising
(a) providing a substrate comprising a plurality of attached proteins corresponding to at least a portion of a proteome, wherein each attached protein comprises a unique spatial address on the substrate, wherein the identity of the protein at each said spatial address is unknown;
(b) determining the identity of the proteins at each spatial address;
(c) applying a ligand to the substrate;
(d) determining one or more spatial addresses where the ligand binds, and optionally, determining one or more spatial addresses where the ligand does not bind; and
(e) identifying at least one protein on the array to which the ligand binds
